SEQUENCE LISTING (4 sequences)

SEQ ID NO: 1 - DNA, 1152 bp, Homo sapiens
  source 1..1152: mol_type = genomic DNA
atgctgtcccgaaagaaaaccaaaaacgaagtgtccaagccggccgaggtgcaggggaagtacgtgaagaaggagacgtcgcctctgcttcggaatcttatgccttcattcatccggcatggtccaacaattccaagacgaactgatatctgtcttccagattcaagccctaatgccttttcaacttctggagatgtagtttcaagaaaccagagtttccttagaactccaattcaaagaacacctcatgaaataatgagaagagaaagcaacagattatctgcaccttcttatcttgccagaagtctagcagatgtccctagagagtatggttcttctcagtcatttgtaacggaagttagttttgctgttgaaaatggagactctggttcccgatattattattcagacaatttttttgatggtcagagaaagcggccacttggagatcgtgcacatgaagactacagatattatgaatacaaccatgatctcttccaaagaatgccacagaatcaggggaggcatgcttcaggtattgggagagttgctgctacatctttaggaaatttgactaaccatggttctgaagatttaccccttcctcctggctggtctgtggactggacaatgagagggagaaaatattatatagatcataacacaaatacaactcactggagccatcctcttgagcgagaaggacttcctcctggatgggaacgagttgagtcatccgaatttggaacctattatgtagatcacacaaataagaaggcccaatacaggcatccctgtgctcctagtgtacctcggtatgatcaaccacctcctgtcacataccagccacagcaaactgaaagaaatcagtcccttctggtacctgcaaatccatatcatactgcagaaattcctgactggcttcaggtttacgcacgagcccctgtgaaatatgaccacattctgaagtgggaactcttccagctggctgacctggatacataccagggaatgctaaagttgctcttcatgaaagaattggagcagattgttaaaatgtatgaagcatacagacaagcccttcttacagagttggaaaaccgaaagcagagacagcagtggtatgcccaacaacatggaaaaaatttttga

SEQ ID NO: 2 - DNA, 21 bp, synthetic construct
  source 1..21: mol_type = other DNA
aagtacgtgaagaaggagacg

SEQ ID NO: 3 - DNA, 21 bp, synthetic construct
  source 1..21: mol_type = other DNA
aagatttaccccttcctcctg

SEQ ID NO: 4 - DNA, 21 bp, synthetic construct
  source 1..21: mol_type = other DNA
aattcctgactggcttcaggt

(19) United States
(12) Patent Application Publication
(10) Pub. No.: US 2025/0283080 A1
(43) Pub. Date: Sep. 11, 2025

(54) GENE THERAPY OF HIPPO SIGNALING IMPROVES HEART FUNCTION IN A CLINICALLY RELEVANT MODEL

(71) Applicants: Baylor College of Medicine, Houston, TX (US); Texas Heart Institute, Houston, TX (US); YAP Therapeutics, Inc., Laguna Hills, CA (US)

(72) Inventors: James F. Martin, Pearland, TX (US); John P. Leach, Pearland, TX (US); Shijie Liu, Houston, TX (US); Sui Zhang, Houston, TX (US)

(73) Assignees: Baylor College of Medicine, Houston, TX (US); Texas Heart Institute, Houston, TX (US); YAP Therapeutics, Inc., Laguna Hills, CA (US)

(21) Appl. No.: 18/876,035

(22) PCT Filed: Jun. 29, 2023
(86) PCT No.: PCT/US2023/069359

§ 371 Date: Dec. 17, 2024

Related U.S. Application Data
(60) Provisional application No. 63/356,673, filed on Jun. 29, 2022.

(51) Int. Cl. C12N 15/113; A61K 48/00; A61P 9/10; C12N 15/86

(52) U.S. Cl. CPC C12N 15/113; A61K 48/0058; A61P 9/10; C12N 15/86; C12N 2310/11; C12N 2310/531; C12N 2320/32; C12N 2750/14143; C12N 2830/008; C12N 2830/48

(57) ABSTRACT

Embodiments of the disclosure include methods and compositions for improving heart function, including reducing the risk of arrhythmia in an individual in need thereof. In particular embodiments, an individual with a need for improved heart function and/or reduced risk of arrhythmia is provided an effective amount of a shRNA molecule that targets the Sav1 gene.

### CROSS-REFERENCE TO RELATED APPLICATIONS

This application claims the benefit of priority to U.S. Provisional Patent Application Ser. No. 63/356,673, filed Jun. 29, 2022, hereby incorporated by reference in its entirety.

### TECHNICAL FIELD

The present disclosure concerns at least the fields of cell biology, molecular biology, and medicine.

### BACKGROUND

The Hippo pathway is activated in the heart in response to physiologic inputs such as changes in extracellular matrix (ECM) composition or mechanical signaling and is maladaptively upregulated in human heart failure (HF) (Leach et al., 2017; Wang et al., 2018). Core Hippo pathway components, including the Mst kinases and the adaptor Salvador (Sav), phosphorylate Lats kinases, which subsequently phosphorylate and inhibit the downstream transcription cofactors Yap and Taz. When Hippo pathway activity is low, Yap and Taz enter the nucleus, where they cooperate with Tead family transcription factors to activate target genes. In adult cardiomyocytes (CMs), high levels of Yap produced via transgene expression induce the transition of cells to a more fetal-like, renewable state by changing genome-wide chromatin accessibility (Monroe et al., 2019). Yap-Tead target genes in CMs include many genes that promote cell cycle progression, including multiple cyclin genes and genes that promote cytoskeletal remodeling and protrusion formation in border zone (BZ) CMs (Morikawa et al., 2015). Because Sav encodes an adaptor rather than a kinase, Sav loss of function moderately inhibits Hippo signaling, which is desirable for human CM renewal therapy (Heallen et al., 2013). Moreover, because there is a single Sav gene in mammalian genomes, it is an attractive target for therapy. Indeed, mild Hippo pathway inhibition via Sav knockdown is a viable strategy to safely treat human HF, as has been suggested by studies in mice (Leach et al., 2017).

Although mouse studies are valuable, they have limitations for translational studies because of the distinctly different cardiovascular anatomy and physiology between rodents and humans. In addition to obvious differences in size, heart rates are much faster in rodents (300-840 beats/min in mice and 330-480 beats/min in rats) than in humans (80-100 beats/min) (Spannbauer et al., 2019; van der Velden et al., 2004).

In contrast, pig hearts share many similarities with human hearts, both in the steady state and after myocardial infarction (MI) (Spannbauer et al., 2019; van der Velden et al., 2004). For example, pig and human hearts have similar contractile indices, as determined by cardiac catheterization measurements (Stubenitsky et al., 1997; Milani-Nejad et al., 2014). Furthermore, pig and human CMs share many characteristics in excitation-contraction coupling. Similar to human CMs, pig CMs predominantly express β-myosin heavy chain, and, similar to the human heart, both stiff N2B and compliant N2BA titin isoforms are expressed in pig myocardium (Milani-Nejad et al., 2014). Pigs also share similar regional cardiac hemodynamic features with humans (McCormick et al., 2016). In diseased pig and human hearts, altered myofilament function is seen after MI (van der Velden et al., 2004), and both pig and human hearts show reduced contractility after MI, which is caused by alterations in Ca2+ handling (van der Velden et al., 2004). Reduced SERCA2a expression has been reported in both species van der Velden et al., 2004). Increased Ca2+ sensitivity is also a common feature in pig and human hearts after MI (Stubenitsky et al., 1997), and the two species share hemodynamic similarities in HF secondary to increased afterload (Gyongyosi et al., 2017). Therefore, the similarities between pig and human hearts make pig models superior to mouse models for studying treatment of cardiac conditions.

A recent study in pigs in which overexpression of miR199a initially improved heart function after MI, resulted in an eventual demise of the pigs two months after viral delivery, most likely due to arrhythmia (Gabisonia et al., 2019). Studies in mice have also shown that overexpression of constitutively active Yap or microRNAs broadly in the heart can be deleterious (Tian et al., 2015; Monroe et al., 2019). Before the present disclosure, it was unknown whether the Hippo pathway's inhibitory regulation of CM proliferation is conserved in large mammals. The present disclosure satisfies the long-felt need for therapeutic CM proliferation and provides methods and compositions for treatment or circumvention of arrhythmia from any cause.

### BRIEF SUMMARY

Chronic human diseases such as HF are difficult to treat, commonly lethal, and associated with an aging population. A characteristic of nonregenerative organs such as the heart is that the functional cell type, such as the CM of the heart, is highly specialized with a limited capacity for self-renewal. The present disclosure demonstrates that using gene therapy to knock down the Hippo pathway in CMs has cardiovascular benefits in a pig model of MI, without deleterious effects of Hippo pathway loss of function in non-CMs, such as cardiac fibroblasts (Xiao et al., 2019). This is the first demonstration that tissue renewal therapy—the approach of inducing tissue renewal by inhibiting an endogenous pathway in poorly regenerative organs—is effective and safe in the translational context of a large animal model.

The examples herein indicate that the Hippo pathway inhibits CM proliferation in pigs, indicating that a similar effect would occur in mammalian hearts, including the human heart. These findings demonstrate that AAV9-Sav-shRNA treatment, for example, induces a steady improvement in heart function in a manner that is safe and effective for treatment of heart disease, particularly in human patients that have experienced myocardial infarction and/or myocardial fibrosis.

In particular embodiments, an individual in need of therapy for a cardiac medical condition is provided an effective amount of one or more nucleic acids, or cells comprising one or more nucleic acids, in which the nucleic acids directly or indirectly provide therapeutic benefit to the individual. In specific embodiments, the nucleic acid is a form that directly or indirectly provides RNA interference, including at least shRNA. In particular embodiments, the shRNA composition targets a member of the Hippo pathway. Although it may target any member of the Hippo pathway, in specific embodiments, the shRNA targets Salvador (Sav1).

In embodiments of the disclosure, there are nucleic acid compositions that target human Sav1, and in specific embodiments the nucleic acid compositions comprise shRNA molecules. In specific embodiments, there are therapeutic compositions that comprise shRNA molecules comprising one or more of SEQ ID NO:2, SEQ ID NO:3, or SEQ ID NO:4. The compositions may or may not be encompassed in a vector, including a viral vector or non-viral vector. In particular embodiments, the shRNA sequences are utilized in a non-integrating vector.

In some embodiments, there is an isolated synthetic nucleic acid composition, comprising SEQ ID NO:2, SEQ ID NO:3, or SEQ ID NO:4 and/or a derivative nucleic acid comprising at least 80% identity to one of SEQ ID NO:2, SEQ ID NO:3, or SEQ ID NO:4. The derivative nucleic acid may be at least 85%, 90%, 95%, 96%, 97%, 98%, or 99% identical to one of SEQ ID NO:2, SEQ ID NO:3, or SEQ ID NO:4.

In certain embodiments, the nucleic acid comprises the sequence of SEQ ID NO:2, SEQ ID NO:3, or SEQ ID NO:4 and further comprises an antisense sequence of SEQ ID NO:2, SEQ ID NO:3, or SEQ ID NO:4 respectively, wherein when the sequence and the antisense sequence are hybridized together to form a duplex structure, the sequence and the antisense sequence are separated by a loop structure.

In specific embodiments, the nucleic acid is at least 43 nucleotides in length or no more than 137 nucleotides in length. In some embodiments, the loop structure is between 5 and 19 nucleotides in length. In particular embodiments, the derivative nucleic acid has 1, 2, 3, 4, or 5 mismatches compared to the respective SEQ ID NO:2, SEQ ID NO:3, or SEQ ID NO:4.

In some embodiments of the composition, the nucleic acid or derivative nucleic acid is comprised in a vector, such as a viral vector or a non-viral vector. The vector may be a non-integrating vector. The vector may be a non-integrating vector that is a lentiviral vector. In some embodiments of the vector, two or more of nucleic acids comprising SEQ ID NO:2, SEQ ID NO:3, or SEQ ID NO:4 are present on the same vector.

In specific embodiments, the expression of the nucleic acid is regulated by a tissue-specific or cell-specific promoter, such as a cardiomyocyte-specific promoter, for example the rat ventricle-specific cardiac myosin light chain 2 (MLC-2v) promoter; cardiac muscle-specific alpha myosin heavy chain (MHC) gene promoter; cardiac cell-specific minimum promoter from −137 to +85 of NCX1 promoter; chicken cardiac troponin T (cTNT), or a combination thereof.

In certain embodiments, two or more of nucleic acids comprising SEQ ID NO:2, SEQ ID NO:3, or SEQ ID NO:4 are present on the same vector. In specific cases, two or more nucleic acids are regulated by the same regulatory sequence or are regulated by a different regulatory sequence.

Methods of the invention include improving systolic function in a human patient who has experienced myocardial infarction, reducing arrhythmias in a human patient with myocardial fibrosis, and promoting capillary formation in heart tissue in a human patient in need thereof. The methods comprise providing an effective amount of a composition encompassed by the disclosure to the individual. The composition may be provided to the individual more than once. The composition may be provided to the individual systemically or locally. In a specific embodiment, the individual is provided an additional therapy for a cardiac condition.

In certain embodiments, there is a kit comprising a composition as encompassed by the disclosure, wherein the composition is housed in a suitable container.

In particular embodiments, the methods of the invention comprise providing to the individual a therapeutically effective amount of a shRNA that targets Salvador (Sav1). In a specific embodiment, the shRNA is provided to the individual in the AAV9 vector.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present disclosure, reference is now made to the following descriptions taken in conjunction with the accompanying drawings.

FIGS. 1A-1J shows improved heart function in Sav knockdown pigs after myocardial infarction (MI). FIG. 1A is a schematic showing one example of the timing of virus injection, EdU injections, and echocardiography (echo) studies in a pig model of ischemia-reperfusion-induced MI. FIG. 1B-1D are echo results showing the ejection fraction (EF) post-MI (FIG. 1B), and at days 14 and 104 in AAV9-GFP-injected (FIG. 1C) or AAV9-Sav-shRNA-injected pig hearts (FIG. 1D) (combined high and low-dose of AAV9-Sav-shRNA). FIGS. 1E-1F show longitudinal plots of changes in EF related to day 14 (virus injection) (high and low doses separated) (FIG. 1E); bar graph with individual datapoints showing changes in EF on day 104 relative to day 14 (FIG. 1F). FIG. 1G-1I show left ventricular end-diastolic volume (LVEDV) (FIG. 1G); left ventricular end-systolic volume (LVESV) (FIG. 1H); and stroke volume (FIG. 1I) (AAV9-GFP, n=7; AAV9-Sav-shRNA, n=8; high-dose AAV9-Sav-shRNA n=3). Two-way ANOVA with Bonferroni's post-hoc test was used for comparisons in FIGS. 1B, 1E, 1G, 1H, and 1I; one-way ANOVA with Tukey's post-hoc test was used for FIG. 1F; the Mann-Whitney test was used for FIGS. 1C and 1D. * indicates comparison between AAV9-GFP (GFP) and AAV9-Sav-shRNA (Sav); # indicates the comparison between AAV9-GFP and AAV9-Sav-shRNA high dose (Sav-high). * or #P<0.05, ** or ##P<0.01, *** or ###P<0.001. FIG. 1J shows quantification of scar size (AAV9-GFP, n=7; AAV9-Sav-shRNA, n=11). The Mann-Whitney test was used for the comparison. Data are presented as the mean±SEM. *P<0.05.

FIGS. 2A-2E show heart regeneration in Sav knockdown pigs. Echocardiograpphy reveals improved contractile function in hearts injected with AAV9-Sav-shRNA compared to hearts injected with AAV9-GFP at different time points. Day 0 indicates the day of myocardial infarction, and day 14 indicates the day of virus injection. FIG. 2A shows injection fraction at different time points in pigs injected with AAV9-GFP (n=7) or AAV9-Sav-shRNA (n=11:8 pigs received a regular dose, 3 pigs received a high dose). FIG. 2B shows heart weight/body weight for pigs at 90 days after virus injection. FIG. 2C shows change (relative to day 14) in left ventricular end-systolic volume (LVESV). FIG. 2D shows change (relative to day 14) in left ventricular end-diastolic volume (LVEDV). FIG. 2E shows changes in stroke volume at different time points. ANOVA with Bonferroni's post-hoc test was used for comparisons in FIGS. 2A, 2C, 2D, and 2E; the Mann-Whitney test was used for comparisons in FIG. 2B. Data are presented as the mean±SEM. *P<0.05, **P<0.01, ***P<0.001.

Immunofluorescence staining for isolectin B4 is shown in FIG. 3A. For each pig heart, 2 to 4 tile images were quantified (n=5). Quantification of immunofluorescence staining for CD45 is shown in FIG. 3B. For each pig, 5 images were quantified (n=5). The Mann-Whitney test was used for comparisons. Data are presented as the mean±SEM. *P<0.05.

FIG. 4A provides a schematic representation of an example of an experimental timeline. A telemetry device was implanted the day before MI, followed by AAV injection 15 days after MI, and animal sacrifice 45 days after MI. ECG telemetry records were collected throughout the 45 day time period. FIG. 4B shows log transformed PVC frequency in the period after MI. Nonlinear regression (single gray line), extra sum-of-squares F Test were used to compare the fit of one curve fit to all the data sets against the null hypothesis of separate individual curves fit to individual data sets. With P=0.8399, the null is rejected, and a single curve is fit to all data sets. FIG. 4C shows log transformed PVC frequency in the period after treatment. Nonlinear regression (individual curve fit line for each treatment group), extra sum-of-squares F Test were used to compare the fit of one curve fit to all the data sets against the null hypothesis of separate individual curves fit to individual data sets. With P<0.0001, the null is accepted, and individual curves are fit to individual data sets. FIG. 4D demonstrates representative ECG traces with evidence of PVCs (arrows) 2 days after MI, or 10 days after treatment for the indicated treatment group. FIG. 4E shows cumulative daily A-Tach incidence in the period after MI. FIG. 4F shows cumulative daily A-Tach incidence in the period after treatment with AAV-GFP or AAV-Sav-shRNA. FIG. 4G shows generalized linear mixed effects model analysis for A-Tach incidence after MI. There was no difference in the predicted odds of an A-Tach event between the two treatment groups during this period after MI and before AAV injections. FIG. 4H demonstrates generalized linear mixed effects model analysis for A-Tach incidence after AAV injection. There is a significant difference (Pr(>|z|), 0.0066**) in the predicted odds of an A-Tach event between the two treatment groups AAV-GFP (Predicted Odds 0.5, 95% CI [0.04, 0.60]), AAV-Sav-shRNA (Predicted Odds 0.3, 95% CI [0.21, 0.40]). FIG. 4I shows representative ECG traces showing atrial tachycardia at different time points after MI. FIG. 4J provides a representative normal ECG recorded 10 days after AAV-Sav-shRNA injection.

### DETAILED DESCRIPTION

### I. Exemplary Definitions

The use of the terms “a” and “an” and “the” and similar referents in the context of describing the invention (especially in the context of the following claims) are to be construed to cover both the singular and the plural, unless otherwise indicated herein or clearly contradicted by context. The terms “comprising,” “having,” “including,” and “containing” are to be construed as open-ended terms (i.e., meaning “including, but not limited to”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Throughout this specification, unless the context requires otherwise, the words “comprise,” “comprises,” and “comprising” will be understood to imply the inclusion of a stated step or element or group of steps or elements but not the exclusion of any other step or element or group of steps or elements. “Consisting of” means including, and limited to, whatever follows the phrase “consisting of.” Thus, the phrase “consisting of” indicates that the listed elements are required or mandatory, and that no other elements may be present. “Consisting essentially of” means including any elements listed after the phrase, and limited to other elements that do not interfere with or contribute to the activity or action specified in the disclosure for the listed elements. Thus, the phrase “consisting essentially of” indicates that the listed elements are required or mandatory, but that other elements are optional and may or may not be present depending upon whether or not they affect the activity or action of the listed elements.

Reference throughout this specification to “one embodiment,” “an embodiment,” “a particular embodiment,” “a related embodiment,” “a certain embodiment,” “an additional embodiment,” or “a further embodiment” or combinations thereof means that a particular feature, structure, or characteristic described in connection with the embodiment is included in at least one embodiment of the present invention. Thus, the appearances of the foregoing phrases in various places throughout this specification are not necessarily all referring to the same embodiment. Furthermore, the particular features, structures, or characteristics may be combined in any suitable manner in one or more embodiments.

As used herein, the terms “or” and “and/or” are utilized to describe multiple components in combination or exclusive of one another. For example, “x, y, and/or z” can refer to “x” alone, “y” alone, “z” alone, “x, y, and z,” “(x and y) or z,” “x or (y and z),” or “x or y or z.” It is specifically contemplated that x, y, or z may be specifically excluded from an embodiment.

Throughout this application, the term “about” is used according to its plain and ordinary meaning in the area of cell and molecular biology to indicate that a value includes the standard deviation of error for the device or method being employed to determine the value.

As used herein, the term “complementary nucleotide sequence,” also known as an “antisense sequence,” refers to a sequence of a nucleic acid that is completely complementary to the sequence of a “sense” nucleic acid encoding a protein (e.g., complementary to the coding strand of a double-stranded cDNA molecule or complementary to an mRNA sequence). Herein, nucleic acid molecules are provided that comprise a sequence complementary to at least, at most, exactly, or about 15, 16, 17, 18, 19, 20, 21, 22, 23, 24, or 25 nucleotides. An antisense nucleic acid sequence or oligonucleotide may be a sequence of DNA or RNA that can bind to via base-pairing a target “sense” sequence. In some instances, the sense sequence is a nucleic acid encoding a protein, such that the antisense sequence is complementary to the coding strand of a double-stranded cDNA molecule or complementary to an mRNA sequence. The antisense sequence can be fully or partially complementary to the sense sequence. Antisense nucleotide sequences can be designed to specifically hybridize to a particular region of a desired target protein or mRNA to interfere with replication, transcription, or translation. An antisense sequence can be complementary to any length of sense sequence, for example, to at least, at most, exactly, or about 15, 16, 17, 18, 19, 20, 21, 22, 23, 24, or 25 nucleotides.

In particular embodiments, when antisense nucleotides (nucleic acids) or siRNA's (small inhibitory RNA) (processed from the shRNA) bind to a target sequence, a particular antisense or small inhibitory RNA (siRNA) sequence is substantially complementary to the target sequence, and thus will specifically bind to a portion of an mRNA-encoding polypeptide. As such, typically the sequences of those nucleic acids will be highly complementary to the mRNA target sequence, and will have no more than 1, 2, 3, 4, 5, 6, 7, 8, 9, or 10 base mismatches throughout the sequence. In many instances, it may be desirable for the sequences of the nucleic acids to be exact matches, i.e., be completely complementary to the sequence to which the oligonucleotide specifically binds, and therefore have zero mismatches along the complementary stretch. Highly complementary sequences will typically bind quite specifically to the target sequence region of the mRNA and will therefore be highly efficient in reducing, and/or even inhibiting, the translation of the target mRNA sequence into polypeptide product. See, e.g., U.S. Pat. No. 7,416,849.

Substantially complementary oligonucleotide sequences may be greater than about 80% complementary (or % exact-match) to the corresponding mRNA target sequence to which the oligonucleotide specifically binds, and will, more preferably, be greater than about 85% complementary to the corresponding mRNA target sequence to which the oligonucleotide specifically binds. In certain aspects, as described above, it will be desirable to have even more substantially complementary oligonucleotide sequences for use in the practice of the invention, and in such instances, the oligonucleotide sequences will be greater than about 90% complementary to the corresponding mRNA target sequence to which the oligonucleotide specifically binds, and may in certain embodiments be greater than about 95% complementary to the corresponding mRNA target sequence to which the oligonucleotide specifically binds, up to and including at least, at most, exactly, or between any two of 96%, 97%, 98%, 99%, and even 100% exact match complementary to the target mRNA to which the designed oligonucleotide specifically binds. See, e.g., U.S. Pat. No. 7,416,849. Percent similarity or percent complementary of any nucleic acid sequence may be determined, for example, by utilizing any computer programs known in the art.

The terms “inhibitory nucleic acid” and “inhibitory oligonucleotide” are used interchangeably and refer to a molecule that knocks down expression of a target gene by preventing translation of the corresponding mRNA. As discussed above, expression is inhibited by sequence-specific binding of the inhibitory nucleic acid to its target. Certain inhibitory RNAs, such as short hairpin RNA (shRNA) and short interfering RNA (siRNA), utilize sequence complementarity to target an mRNA for destruction. When appropriately targeted via its nucleotide sequence to a specific mRNA in cells, the inhibitory RNA specifically suppresses target gene expression, reducing the cellular level of the corresponding target mRNA and decreasing the level of protein encoded by such mRNA.

Inhibitory nucleic acids can be single-stranded or double-stranded. Examples of inhibitory nucleic acids include antisense DNA and RNA oligonucleotides, siRNA, shRNA, and micro-RNA. As used herein, the term “knock-down” or “knock-down technology” refers to a technique of gene silencing in which the expression of a target gene or gene of interest is reduced as compared to the gene expression prior to the introduction of an inhibitory RNA, such as an shRNA, which can lead to the inhibition of production of the target gene product. For example, the expression may be reduced by at least, at most, exactly, or between any two of 0.1, 0.5, 1, 5, 10, 15, 20, 25, 30, 35, 40, 45, 50, 55, 60, 65, 70, 75, 80, 85, 90, 95, or even 99%. The expression may be reduced by any amount (%) within those intervals, such as for example, 2-4, 11-14, 16-19, 21-24, 26-29, 31-34, 36-39, 41-44, 46-49, 51-54, 56-59, 61-64, 66-69, 71-74, 76-79, 81-84, 86-89, 91-94, 96, 97, 98, or 99. Reduction of gene expression can be statistically significant, as measured, for example, by a student's T test or other known statistical method, compared to unaltered or wild-type gene expression. Knock-down of gene expression can be directed by techniques known in the art, such as by the use of inhibitory RNA or by the use of genomic editing, such as by CRISPR or TALENs.

As used herein, the term “knock-down” or “knock-down technology” refers to a technique of gene silencing in which the expression of a target gene or gene of interest is reduced as compared to the gene expression prior to the introduction of the shRNA, which can lead to the inhibition of production of the target gene product. The term “reduced” is used herein to indicate that the target gene expression is lowered by 0.1-100%. For example, the expression may be reduced by at least, at most, exactly, or between any two of 0.5, 1, 5, 10, 15, 20, 25, 30, 35, 40, 45, 50, 55, 60, 65, 70, 75, 80, 85, 90, 95, or even 99%. The expression may be reduced by any amount (%) within those intervals, such as for example, 2-4, 11-14, 16-19, 21-24, 26-29, 31-34, 36-39, 41-44,46-49, 51-54, 56-59, 61-64, 66-69, 71-74, 76-79, 81-84, 86-89, 91-94, 96, 97, 98 or 99. Knock-down of gene expression can be directed by the use of siRNAs or shRNAs.

As used herein, the term “nucleotide sequence” or “nucleic acid” refers to a polymer of DNA or RNA having a combination of purine and pyrimidine bases, sugars, and covalent linkages between nucleosides including a phosphate group in a phosphodiester linkage. A nucleic acid can be single-stranded or double-stranded, and will optionally contain synthetic, non-natural, or altered nucleotide bases capable of incorporation into DNA or RNA polymers. The term “polynucleotide” is used interchangeably with the term “oligonucleotide.” “Nucleotide sequence” and “nucleic acid sequence” are terms referring to a sequence of nucleotides in a polynucleotide molecule. The term “nucleotide sequence” is interchangeable with “nucleic acid sequence” unless otherwise clearly stated. Where a sequence comprising thymine is provided, it should be understood that the corresponding RNA sequence comprises uracil at the positions indicated as thymine.

In some cases, nucleic acid analogs are included that may have alternate backbones or non-natural internucleoside linkages, comprising, for example, modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like. See, e.g., U.S. Pat. No. 7,410,944. Examples of modified phosphorous-containing backbones include phosphoramide, phosphorothioate, phosphorodithioate, chiral phosphorothioate, O-methylphophoroamidite phosphotriester, aminoalkylphosphotriester, alkyl phosphonate, thionoalkylphosphonate, phosphinate, phosphoramidate, thionophosphoramidate, thionoalkylphosphotriester, boranophosphate, and various salt forms thereof. Examples of the non-phosphorous containing backbones described above are known in the art, e.g., U.S. Pat. No. 5,677,439, each of which is herein incorporated by reference. Other analog nucleic acids include those with positive backbones, non-ionic backbones, and non-ribose backbones, including those described in U.S. Pat. Nos. 5,235,033 and 5,034,506. Modification of the ribose-phosphate backbone may facilitate the addition of moieties such as labels or increase the stability and half-life of such molecules in physiological environments.

Nucleic acids can contain substituted or modified sugar moieties, e.g., 2′-O-methoxyethyl sugar moieties or carbocyclic sugars. Nucleic acids can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g., 6-methyluridine), 2-thiouridine, 4-thiouridine, 5-(carboxyhydroxy methyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyl uridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyl adenosine, beta-D-mannosylqueosine, uridine-5-oxyacetic acid, 2-thiocytidine, threonine derivatives, and the like. See, e.g., U.S. Pat. No. 7,410,944.

As used herein, the term “operably-linked” refers to the association of nucleic acid sequences on a polynucleotide so that the function of one of the sequences is affected by another. For example, a regulatory DNA sequence is said to be “operably linked to” a DNA sequence that codes for an RNA (“an RNA coding sequence” or “shRNA encoding sequence”) or a polypeptide if the two sequences are situated such that the regulatory DNA sequence affects expression of the coding DNA sequence (i.e., that the coding sequence or functional RNA is under the transcriptional control of the promoter). Coding sequences can be operably-linked to regulatory sequences in sense or antisense orientation. An RNA coding sequence refers to a nucleic acid that can serve as a template for synthesis of an RNA molecule such as an siRNA and an shRNA. Preferably, the RNA coding region is a DNA sequence.

As used herein, the term “pharmaceutically acceptable” means that the compound is physiologically acceptable, does not cause adverse reactions, and exerts no inhibitory effects on the action of an active ingredient when it is administered to a patient.

As used herein, the term “promoter” refers to a nucleotide sequence, usually upstream (5′) to its coding sequence, which directs and/or controls the expression of the coding sequence by providing the recognition for RNA polymerase and other factors required for proper transcription. “Promoter” includes a minimal promoter that is a short DNA sequence comprised of a TATA-box and other sequences that serve to specify the site of transcription initiation, to which regulatory elements are added for control of expression. “Promoter” also refers to a nucleotide sequence that includes a minimal promoter plus regulatory elements capable of controlling the expression of a coding sequence or functional RNA. This type of promoter sequence consists of proximal and more distal upstream elements, the latter elements often referred to as enhancers. Accordingly, an “enhancer” is a DNA sequence that stimulates promoter activity and may be an innate element of the promoter or a heterologous element inserted to enhance the level or tissue specificity of a promoter. It is capable of operating in both orientations (sense or antisense) and is capable of functioning even when moved either upstream or downstream from the promoter. Both enhancers and other upstream promoter elements bind sequence-specific DNA-binding proteins that mediate their effects. Promoters may be derived in their entirety from a native gene or may be composed of different elements derived from different promoters found in nature, or may even be comprised of synthetic DNA segments. A promoter may also contain DNA sequences that are involved in the binding of protein factors that control the effectiveness of transcription initiation in response to physiological or developmental conditions. Any promoter known in the art which regulates the expression of the shRNA or RNA coding sequence is envisioned in the practice of the invention.

As used herein, the term “reporter element” or “marker” means a polynucleotide that encodes a polypeptide capable of being detected in a screening assay. Examples of polypeptides encoded by reporter elements include, but are not limited to, lacZ, GFP, luciferase, and chloramphenicol acetyltransferase. See, e.g., U.S. Pat. No. 7,416,849. Many reporter elements and marker genes are known in the art and envisioned for use in the inventions disclosed herein.

As used herein, the term “RNA transcript” refers to the product resulting from RNA polymerase catalyzed transcription of a DNA sequence. “Messenger RNA transcript” (“mRNA”) refers to the RNA that is without introns and that can be translated into protein by the cell.

As used herein, the terms “small interfering RNA” or “short interfering RNA” or “siRNA” refer to an RNA duplex of nucleotides that is targeted to a desired gene and is capable of inhibiting the expression of a gene with which it shares homology. The RNA duplex comprises two complementary single-stranded RNAs of at least, at most, or exactly 15, 16, 17, 18, 19, 20, 21, 22, 23, 24, or 25 nucleotides that form at least, at most, or exactly 15, 16, 17, 18, 19, 20, 21, 22, 23, 24, or 25 base pairs and possess 3′ overhangs of two nucleotides. The RNA duplex is formed by the complementary pairing between two regions of a RNA molecule. siRNA is “targeted” to a gene in that the nucleotide sequence of the duplex portion of the siRNA is complementary to a nucleotide sequence of the targeted gene. In some embodiments, the length of the duplex of siRNAs is less than 30 nucleotides. The duplex can be at least, at most, or exactly 29, 28, 27, 26, 25, 24, 23, 22, 21, 20, 19, 18, 17, 16, 15, 14, 13, 12, 11 or 10 nucleotides in length. The length of the duplex can be 17-25 nucleotides in length. The duplex RNA can be expressed in a cell from a single construct.

As used herein, the term “shRNA” (small hairpin RNA) refers to an RNA duplex wherein a portion of the siRNA is part of a hairpin structure (shRNA). In addition to the duplex portion, the hairpin structure may contain a loop portion positioned between the two sequences that form the duplex. The loop can vary in length. In some embodiments the loop is at least, at most, or exactly 5, 6, 7, 8, 9, 10, 11, 12 or 13 nucleotides in length. The hairpin structure can also contain 3′ or 5′ overhang portions. In some aspects, the overhang is a 3′ or 5′ overhang at least, at most, or exactly 0, 1, 2, 3, 4 or 5 nucleotides in length. In one aspect of this invention, a nucleotide sequence in the vector serves as a template for the expression of a small hairpin RNA, comprising a sense region, a loop region, and an antisense region. Following expression, the sense and antisense regions form a duplex. It is this duplex, forming the shRNA, which hybridizes to, for example, the Sav1 mRNA and reduces expression of Sav1.

The terms “subject” and “individual” and “patient” are used interchangeably and typically comprise a human.

As used herein, the phrase “subject in need thereof” or “individual in need thereof” refers to a subject or individual that suffers or is at a risk of suffering (e.g., pre-disposed such as genetically pre-disposed, or subjected to environmental conditions that pre-dispose, etc.) from a cardiac symptom, disease, or condition. In certain embodiments, a subject is successfully “treated” for a disease or disorder according to the methods provided herein if the subject shows, e.g., total, partial, or transient alleviation or elimination of one or more symptoms associated with the disease or disorder.

As used herein, the term “treating” refers to ameliorating at least one symptom of, curing, and/or preventing the development of a disease or disorder such as for example, but not limited to, a cardiac medical condition, including arrhythmia, myocardial infarction, ischemic heart disease, heart failure, cardiomyopathy, etc.

As used herein, the term “vector” refers to any viral or non-viral vector, as well as any plasmid, cosmid, phage or binary vector in double or single stranded linear or circular form that may or may not be self-transmissible or mobilizable, and that can transform prokaryotic or eukaryotic host cells either by integration into the cellular genome or which can exist extrachromosomally (e.g., autonomous replicating plasmid with an origin of replication). Any vector known in the art is envisioned for use in the practice of this invention.

### II. General Embodiments

Embodiments of the disclosure concern methods and compositions for improving cardiac function in an individual in need thereof. For example, the individual may have myocardial fibrosis, which can lead to HF. Myocardial fibrosis results from excessive myofibroblast activity and accumulation of extracellular matrix in cardiac tissue. Myocardial fibrosis can be caused, for example, by amyloidosis, hypertensive heart disease, hypertrophic cardiomyopathy, idiopathic dilated cardiomyopathy, myocardial infarction, myocarditis, and sarcoidosis. In a specific embodiment, the individual has arrhythmia associated with myocardial infarction.

Examples provided herein demonstrate cardiovascular benefits without deleterious effects, including at least improvement of systolic function, promotion of vascularity, and treatment of arrhythmia associated with MI. In specific embodiments, CMs are regenerated in these treated hearts without concomitant or subsequent loss of function of cells that are not CMs.

There is demonstrated herein a unique, but exemplary, set of three short hairpin RNAs (shRNA) that specifically target the Hippo pathway member Salvador (Sav1). In particular embodiments, the shRNAs provide selective reduction in Sav1 mRNA levels. In specific embodiments, the shRNAs can be delivered using a vector that has tropism for the heart, such as an AAV9 (Adeno Associated Virus serotype 9) vector that has tropism for the heart.

### III. Salvador

In particular embodiments, the Hippo pathway member Salvador (salvador family WW domain containing protein 1) is targeted with shRNA in treatments for cardiac medical conditions. The gene may be referred to as salvador homolog 1, Salv, SAV1, SAV, WW45, or WWP4. A representative nucleic acid is provided at GenBank® Accession No. CR457297.1, and a representative protein sequence is provided at GenBank® Accession No. Q9H4B6.

The gene encodes a protein which includes 2 WW domains (a modular protein domain that mediates specific interactions with protein ligands) and a coiled-coil region. It is ubiquitously expressed in adult tissues. It also includes a SARAH (Sav/Rassf/Hpo) domain at the C terminus (three classes of eukaryotic tumor suppressors that give the domain its name). In the Say (Salvador) and Hpo (Hippo) families, the SARAH domain mediates signal transduction from Hpo via the Say scaffolding protein to the downstream component Wts (Warts); the phosphorylation of Wts by Hpo triggers cell cycle arrest and apoptosis by down-regulating cyclin E, Diap 1, and other targets. The SARAH domain may also be involved in dimerization.

### IV. Examples of Methods of Treatment

In embodiments of the disclosure are methods of improving cardiac function in an individual. The individual may have experienced myocardial infarction, arrhythmia, heart failure, fibrosis of the heart, cardiomyopathy, ischemic cardiomyopathy, myocardial necrosis, dilated cardiomyopathy, diabetic cardiomyopathy, age-related cardiomyopathy, a combination thereof, and so forth.

In specific embodiments, the individual has an arrhythmia. In such cases, the arrhythmia may be of a tachycardia type or it may be of a bradycardia type. The arrhythmia may be atrial arrhythmia or ventricular arrhythmia, and the individual may or may not have experienced myocardial infarction. Types of tachycardias that may be treated include atrial fibrillation, atrial flutter, supraventricular tachycardia, ventricular fibrillation, or ventricular tachycardia. Types of bradycardia that may be treated include Sick sinus syndrome or conduction block. Symptoms of arrhythmia include a fluttering in the chest, a racing heartbeat, a slow heartbeat, chest pain, shortness of breath, anxiety, fatigue, dizziness, lightheadedness, sweating, and/or fainting.

Embodiments of the disclosure include methods of improving systolic function, the method comprising delivering to heart tissue of a human patient who has experienced myocardial infarction an effective amount of a composition comprising at least one inhibitory nucleic acid, wherein the inhibitory nucleic acid targets Salvador. Embodiments of the disclosure include methods of reducing arrhythmia in a human patient with myocardial fibrosis, the method comprising delivering to heart tissue of the human patient an effective amount of a composition comprising at least one inhibitory nucleic acid, wherein the inhibitory nucleic acid targets Salvador. Embodiments of the disclosure include methods of promoting capillary formation in heart tissue, the method comprising delivering to heart tissue of an individual in need thereof, an effective amount of a composition comprising at least one inhibitory nucleic acid, wherein the inhibitory nucleic acid targets Salvador. Embodiments of the disclosure include methods of improving outcomes for an individual having had a myocardial infarction, the method comprising delivering to heart tissue of the human patient an effective amount of a composition comprising at least one inhibitory nucleic acid, wherein the inhibitory nucleic acid targets Salvador. Embodiments of the disclosure include methods of restoring normal sinus rhythm for an individual having had a myocardial infarction, the method comprising delivering to heart tissue of the human patient an effective amount of a composition comprising at least one inhibitory nucleic acid, wherein the inhibitory nucleic acid targets Salvador. Embodiments of the disclosure include methods of reducing the risk of, delaying onset of, or reducing the severity of induction of arrhythmogenesis for an individual having, or having had, a myocardial infarction, the method comprising delivering to heart tissue of the individual an effective amount of a composition comprising at least one inhibitory nucleic acid, wherein the inhibitory nucleic acid targets Salvador. Embodiments of the disclosure include methods of reducing the risk of post-infarct arrhythmia or any kind, including at least refractory ventricular arrhythmia, the method comprising delivering to heart tissue of the individual an effective amount of a composition comprising at least one inhibitory nucleic acid, wherein the inhibitory nucleic acid targets Salvador. Embodiments of the disclosure include methods of reducing the frequency of ventricular arrhythmia after myocardial infarction, the method comprising delivering to heart tissue of the individual an effective amount of a composition comprising at least one inhibitory nucleic acid, wherein the inhibitory nucleic acid targets Salvador.

Embodiments of the disclosure include methods of treating, preventing, or reducing the risk of arrhythmia in a human patient, the method comprising (a) identifying a need for treatment, prevention, or reduction of the risk of arrhythmia in a human patient; and (b) delivering to heart tissue of the human patient an effective amount of a composition comprising at least one inhibitory nucleic acid, wherein the inhibitory nucleic acid targets Salvador. In specific embodiments, the human patient has experienced myocardial infarction or is at risk for myocardial infarction.

In methods encompassed herein, the nucleic acid may be provided to the individual once or more than once. The nucleic acid compositions of the disclosure may be provided to the individual systemically or locally, including by injection in and/or near the heart. The delivery may occur upon the diagnosis of a need for improved heart function, such as improved systolic function, promotion of capillary formation in heart tissue, and/or reduction of arrhythmia. Improvement in heart function in an individual subjected to treatment by a method of the invention is relative to heart function in the individual prior to treatment by a method of the invention. Heart function can be assessed, for example, by measuring ejection fraction, diastolic volume, systolic volume, left ventricular end-diastolic volume, left ventricular end-systolic volume, left ventricular systolic function, diastolic function, stroke volume, cardiac rhythm, or combinations thereof.

In a treatment context, reduction of arrhythmia in a patient is assessed as frequency and/or number of arrhythmias in a patient subjected to treatment by a method of the invention, relative to frequency and/or number of arrhythmias in the patient prior to treatment by a method of the invention. In a prevention context, reduction of arrhythmia in a patient is assessed as frequency and/or number of arrhythmias in a patient subjected to treatment by a method of the invention, relative to frequency and/or number of arrhythmias in an individual having comparable heart function to the patient, which individual has not been subjected to treatment by a method of the invention.

In some embodiments, the individual is one at risk for arrhythmia, such as one at risk for arrhythmia during and/or following a myocardial infarction. In specific embodiments, the individual is at risk compared to the general population, such as being at risk because of having a prior history of arrhythmia, advanced age, general health, having one or more genetic factors, having a history of personal and/or familial heart disease, a personal history of arrhythmia, a family history of arrhythmia, and so forth. In specific embodiments, the individual is at risk for having arrhythmia because of having myocardial fibrosis and/or having experienced myocardial infarction.

In specific embodiments of the methods, the inhibitory nucleic acid has, or is encoded by a sequence having, at least 80% identity to a nucleotide sequence selected from the group consisting of SEQ ID NO: 2 (aagtacgtga agaaggagac g), SEQ ID NO: 3 (aagatttacc ccttcctcct g), and SEQ ID NO: 4 (aattcctgac tggcttcagg t). In particular methods, the composition comprises (i) an inhibitory nucleic acid having, or encoded by a sequence having, at least 80% identity to SEQ ID NO: 2, (ii) an inhibitory nucleic acid having, or encoded by a sequence having, at least 80% identity to SEQ ID NO: 3, or (iii) an inhibitory nucleic acid having, or encoded by a sequence having, at least 80% identity to SEQ ID NO: 4. The inhibitory nucleic acid may have, or may be encoded by a sequence having, at least, at most, exactly, or between any two of 85%, 90%, 95%, 96%, 97%, 98%, or 99% identity to a sequence selected from the group consisting of SEQ ID NO: 2, SEQ ID NO: 3, and SEQ ID NO: 4. The inhibitory nucleic acid may have a sequence, or is encoded by a sequence, selected from the group consisting of SEQ ID NO: 2, SEQ ID NO: 3, and SEQ ID NO: 4. In specific embodiments, mixtures of inhibitory nucleic acids that have a sequence, or is encoded by a sequence, selected from the group consisting of SEQ ID NO: 2, SEQ ID NO: 3, and SEQ ID NO: 4 may be utilized. In some embodiments, the composition comprises a derivative nucleic acid SEQ ID NO: 2, SEQ ID NO: 3, and SEQ ID NO: 4 that has at least, at most, or exactly 1, 2, 3, 4, or 5 mismatches compared to the respective SEQ ID NO.

In certain methods of the disclosure, the inhibitory nucleic acid is of any kind and may be an antisense DNA molecule, an RNA, or a short hairpin RNA (shRNA), for example. The inhibitory nucleic acid may or may not be of a particular length, such as at least, at most, or exactly 25, 26, 27, 28, 29, 30, 31, 32, 33, 34, 35, 36, 37, 38, 39, 40, 41, 42, 43, 44, 45, 46, 47, 48, 49, or 50 or more nucleotides in length. In specific embodiments, the inhibitory nucleic acid is a shRNA that is at least 43 nucleotides in length. The inhibitory nucleic acid may or may not be less than a certain number of nucleotides in length, including less than 150, 149, 148, 147, 146, 145, 144, 143, 142, 141, 140, 139, 138, 137, 136, 135, 134, 133, 132, 131, 130, 129, 128, 127, 126, 125, and so forth nucleotides in length. In specific cases, the inhibitory nucleic acid is an shRNA that is less than 138 nucleotides in length.

In embodiments of methods in which an shRNA is utilized as the inhibitory nucleic acid, the shRNA may comprise a loop structure of a certain length. In specific cases, the shRNA may comprise a loop structure of between about 5 and about 19 nucleotides in length. The loop structure may be at least, at most, or exactly 5, 6, 7, 8, 9, 10, 11, 12, 13, 14, 15, 16, 17, 18, or 19 nucleotides in length.

In particular embodiments of the methods, the inhibitory nucleic acid or a nucleotide sequence encoding the inhibitory nucleic acid may be comprised in a nucleic acid construct. In specific embodiments, a nucleotide sequence encoding the inhibitory nucleic acid as an RNA is utilized. In specific embodiments, a nucleotide sequence encoding the RNA is expressed in cardiomyocytes, such as the nucleotide sequence encoding the RNA being operably linked to a tissue-specific promoter. One specific promoter includes the cardiac troponin T promoter, rat ventricle-specific cardiac myosin light chain 2 (MLC-2v) promoter; cardiac muscle-specific alpha myosin heavy chain (MHC) gene promoter; cardiac cell-specific minimum promoter from −137 to +85 of NCX1 promoter; chicken cardiac troponin T (cTNT); Nppa promoter (such as for atrial cardiomyocyte specific expression), or a combination thereof. In specific embodiments of the methods, the nucleotide sequence encoding the RNA is comprised in a nucleic acid construct that may or may not comprise a post-transcriptional regulatory element, such as a woodchuck hepatitis virus post-transcriptional regulatory element (WPRE). In some embodiments, the nucleic acid construct may comprise sequences encoding a 3′ microRNA-30 sequence and a 5′ microRNA-30 sequence. The nucleic acid construct may comprise 5′ and 3′ inverted terminal repeats, in specific embodiments.

In specific embodiments of the methods, a nucleotide sequence encoding the inhibitory nucleic acid is comprised in a vector of any kind, including a non-viral vector, a non-integrating vector, a viral vector, and so forth. The viral vector may be adenoviral, adeno-associated viral (AAV), a lentiviral vector, or retroviral, for example. In specific embodiments, the inhibitory nucleic acid is comprised in an AAV vector. The AAV vector can be of any serotype, including, for example, AAV2, AAV6, AAV7, AAV8, and AAV9. In one embodiment, an AAV9 vector is employed. Non-viral vectors include plasmids, transposons, and so forth.

In specific embodiments of the methods, the methods utilize a composition that comprises a nucleic acid construct comprising: (i) a nucleic acid having the nucleotide sequence set forth in SEQ ID NO: 2, (ii) a nucleic acid having the nucleotide sequence set forth in SEQ ID NO: 3, and (iii) a nucleic acid having the nucleotide sequence set forth in SEQ ID NO: 4; wherein nucleic acids (i)-(iii) are operably linked to a promoter. Nucleotide sequences encoding two or more inhibitory nucleic acids may or may not be comprised in a single nucleic acid construct. In aspects wherein the two or more inhibitory nucleic acids are comprised in a single nucleic acid construct, they may or may not be regulated by a single promoter.

In some embodiments of the methods, the individual is provided an effective amount of a therapy other than the therapies encompassed herein. In specific embodiments, the additional therapies can include anti-arrhythmic drugs, such as amiodarone, flecainide, ibutilide, lidocaine, procainamide, popafenone, quinidine, and/or tocainide); calcium channel blockers; beta blockers, and/or anti-coagulants.

### V. Nucleic Acids that Target Sav1

In particular embodiments, there are one or more nucleic acids that target Sav1 such that expression of Sav1 is detectably reduced. The nucleic acids that target Sav1 may be considered inhibitory nucleic acids. The nucleic acids may be DNA or RNA, but in specific embodiments the nucleic acids are RNA, such as shRNA.

In some embodiments, the nucleic acids target human Sav1. One example of human Sav1 is at GenBank® Accession No. NM_021818.4 and is provided below:

(SEQ ID NO: 1)
ATGCTGTCCCGAAAGAAAACCAAAAACGAAGTGTCCAAGCCGGCCGAGG
TGCAGGGGAAGTACGTGAAGAAGGAGACGTCGCCTCTGCTTCGGAATCT
TATGCCTTCATTCATCCGGCATGGTCCAACAATTCCAAGACGAACTGAT
ATCTGTCTTCCAGATTCAAGCCCTAATGCCTTTTCAACTTCTGGAGATG
TAGTTTCAAGAAACCAGAGTTTCCTTAGAACTCCAATTCAAAGAACACC
TCATGAAATAATGAGAAGAGAAAGCAACAGATTATCTGCACCTTCTTAT
CTTGCCAGAAGTCTAGCAGATGTCCCTAGAGAGTATGGTTCTTCTCAGT
CATTTGTAACGGAAGTTAGTTTTGCTGTTGAAAATGGAGACTCTGGTTC
CCGATATTATTATTCAGACAATTTTTTTGATGGTCAGAGAAAGCGGCCA
CTTGGAGATCGTGCACATGAAGACTACAGATATTATGAATACAACCATG
ATCTCTTCCAAAGAATGCCACAGAATCAGGGGAGGCATGCTTCAGGTAT
TGGGAGAGTTGCTGCTACATCTTTAGGAAATTTGACTAACCATGGTTCT
GAAGATTTACCCCTTCCTCCTGGCTGGTCTGTGGACTGGACAATGAGAG
GGAGAAAATATTATATAGATCATAACACAAATACAACTCACTGGAGCCA
TCCTCTTGAGCGAGAAGGACTTCCTCCTGGATGGGAACGAGTTGAGTCA
TCCGAATTTGGAACCTATTATGTAGATCACACAAATAAGAAGGCCCAAT
ACAGGCATCCCTGTGCTCCTAGTGTACCTCGGTATGATCAACCACCTCC
TGTCACATACCAGCCACAGCAAACTGAAAGAAATCAGTCCCTTCTGGTA
CCTGCAAATCCATATCATACTGCAGAAATTCCTGACTGGCTTCAGGTTT
ACGCACGAGCCCCTGTGAAATATGACCACATTCTGAAGTGGGAACTCTT
CCAGCTGGCTGACCTGGATACATACCAGGGAATGCTAAAGTTGCTCTTC
ATGAAAGAATTGGAGCAGATTGTTAAAATGTATGAAGCATACAGACAAG
CCCTTCTTACAGAGTTGGAAAACCGAAAGCAGAGACAGCAGTGGTATGC
CCAACAACATGGAAAAAATTTTTGA

In one embodiment, the inhibitory RNA has or is encoded by the nucleotide sequence (SEQ ID NO: 2). In one embodiment, the inhibitory RNA has or is encoded by the nucleotide sequence (SEQ ID NO: 3). In one embodiment, the inhibitory RNA has or is encoded by the nucleotide sequence (SEQ ID NO: 4).

In one embodiment, the shRNA is a “hairpin” or stem-loop RNA molecule, comprising a sense region, a loop region and an antisense region complementary to the sense region. In other embodiments the inhibitory RNA is an siRNA comprising two distinct, complementary RNA molecules (strands) that are non-covalently associated via base pairing to form a duplex. See, e.g., U.S. Pat. No. 7,195,916.

In particular cases, the inhibitory RNA is an shRNA. In particular cases, shRNA is a single-stranded RNA molecule that forms a stem-loop structure in vivo, and it may be from about 40-135 nucleotides in length. In specific embodiments, the length is from about 40-135, 40-120, 40-100, 40-80, 40-75, 40-50, 50-135, 50-120, 50-100, 50-80, 50-75, 50-60, 75-135, 75-120, 75-100, 75-90, 75-80, 90-135, 90-120, 90-100, 100-135, 110-135, 110-125, or 125-135 nucleotides in length. The shRNA is a single-stranded RNA molecule that forms a stem-loop structure in vivo, and it may be from at least, at most, exactly, or about 40, 41, 42, 43, 44, 45, 46, 47, 48, 49, 50, 51, 52, 53, 54, 55, 56, 57, 58, 59, or 60 nucleotides in length to 120, 121, 122, 123, 124 125, 126, 127, 128, 129, 130, 131, 132, 133, 134, 135, 136, 137, 138, 139 or 140 (nt) in length. In specific embodiments, the duplex portion of the stem-loop structure can be less than 30 nucleotides in length, such as at least, at most, or exactly 10, 11, 12, 13, 14, 15, 16, 17, 18, 19, 20, 21, 22, 23, 24, 25, 26, 27, 28, or 29 nucleotides in length, including ranges within these lengths. In at least certain cases, a 5- to 19-nucleotide loop (including 5, 6, 7, 8, 9, 10, 11, 12, 13, 14, 15, 16, 17, 18, or 19 nucleotides) connects the two complementary 19-29-nucleotide-long RNA fragments (including 19, 20, 21, 22, 23, 24, 25, 26, 27, 28, or 29 nucleotides) that create the double-stranded stem by base pairing. The shRNA can further comprise an overhang region. Such an overhang may be a 3′ overhang region or a 5′ overhang region. The overhang region may be, for example, at least, at most, or exactly 1, 2, 3, 4, 5, or 6 nucleotides in length. Transcription and synthesis of shRNA in vivo is directed by Pol III promoter, and then the resulting shRNA is cleaved by Dicer, an RNase III enzyme, to generate mature siRNA. The mature siRNA enters the RISC complex. Thus, in specific embodiments, shRNA for inhibition of Sav1 expression in accordance with the present disclosure contains both sense and antisense nucleotide sequences.

In certain embodiments, the nucleic acid comprises the sequence of SEQ ID NO:2 (or, respectively, SEQ ID NO:3 or 4) and further comprises an antisense sequence of SEQ ID NO:2 (or, respectively, SEQ ID NO:3 or 4), wherein when the sequence and the antisense sequence are hybridized together to form a duplex structure, the sequence and the antisense sequence are separated by a loop structure.

In a one embodiment, the nucleic acid construct comprises a polynucleotide sequence encoding an shRNA operably linked to a promoter. In one embodiment, the shRNA comprises a first segment, a second segment located immediately 3′ of the first segment, and a third segment located immediately 3′ of the second segment, wherein the first and third segments can each be less than 30 base pairs in length and can each be more than 10 base pairs in length. The first segment and the third segment are complementary to one another, one comprising an antisense sequence and the other comprising a sense sequence, relative to a target sequence. The second segment, located immediately 3′ of the first segment, encodes a loop structure.

In specific embodiments, the inhibitory nucleic acid has, or is encoded by a sequence having, at least, at most, exactly, or between any two of 80%, 85%, 90%, 91%, 92%, 93%, 94%, 95%, 96%, 97%, 98%, or 99% identity to a nucleotide sequence selected from the group consisting of SEQ ID NO: 2, SEQ ID NO: 3, and SEQ ID NO: 4. In some embodiments, the composition comprises a derivative nucleic acid of SEQ ID NO: 2, SEQ ID NO: 3, and SEQ ID NO: 4 that has 1, 2, 3, 4, or 5 mismatches compared to the respective SEQ ID NO.

When appropriately targeted via its nucleotide sequence to a specific mRNA in cells, the shRNA specifically suppresses gene expression of Sav1. In at least some cases, shRNAs can reduce the cellular level of specific mRNAs and decrease the level of proteins coded by such mRNAs. shRNAs utilize sequence complementarity to target an mRNA for destruction and are sequence-specific. Thus, they can be highly target-specific, and in mammals have been shown to target mRNAs encoded by different alleles of the same gene.

In specific embodiments, an shRNA corresponding to a region of a target gene to be down-regulated or knocked-down is expressed in the cell. The shRNA duplex may be substantially identical (for example, at least, at most, exactly, or about 80%, 81%, 82%, 83%, 84%, 85%, 86%, 87%, 88%, 89%, 90%, 91%, 92%, 93%, 94%, 95%, 96%, 97%, 98%, or 99% identical) in sequence to the sequence of the gene targeted for down regulation. In specific embodiments, there are no more than 5 mismatches between the sequence of the shRNA and the target Sav1 sequence. In specific embodiments, a minimum of 18 bp homology is utilized for the region of complementarity between the shRNA sequence and its target. In particular embodiments, specific assays are utilized to test suitable mismatches for the shRNA and its target. In certain embodiments, an algorithm may be employed to identify suitable mismatches for the shRNA and its target.

Thus, it should be noted that full complementarity between the target sequence and the shRNA is not required. That is, the resultant antisense siRNA (following processing of the shRNA) is sufficiently complementary with the target sequence. The sense strand is substantially complementary with the antisense strand to anneal (hybridize) to the antisense strand under biological conditions.

In particular, the complementary polynucleotide sequence of shRNA can be designed to specifically hybridize to a particular region of a desired target protein or mRNA to interfere with replication, transcription, or translation. The term “hybridize” or variations thereof, refers to a sufficient degree of complementarity or pairing between an antisense nucleotide sequence and a target DNA or mRNA such that stable and specific binding occurs there between. In particular, 100% complementarity or pairing is desirable but not required. Specific hybridization occurs when sufficient hybridization occurs between the antisense nucleotide sequence and its intended target nucleic acids in the substantial absence of non-specific binding of the antisense nucleotide sequence to non-target sequences under predetermined conditions, e.g., for purposes of in vivo treatment, preferably under physiological conditions. Preferably, specific hybridization results in the interference with normal expression of the gene product encoded by the target DNA or mRNA. In the context of nucleic acids, the terms “hybridize,” “bind,” “target,” or variations thereof refer to a sufficient degree of complementarity or base pairing between a complementary or inhibitory nucleic acid sequence and a target DNA or mRNA, such that a stable and specific interaction occurs between them. Specific hybridization occurs when sufficient interaction occurs between the complementary or inhibitory nucleotide sequence and its intended target nucleic acids, in the substantial absence of non-specific binding of the complementary or inhibitory nucleotide sequence to non-target sequences under predetermined conditions, preferably under biological or physiological conditions. Preferably, specific hybridization results in inhibition, i.e., the interference with normal expression of the gene product encoded by the target DNA or mRNA. Full complementarity between the target sequence and the inhibitory RNA is not required. The inhibitory nucleotide sequence, e.g., a single-stranded antisense oligonucleotide or the antisense sequence of a double-stranded inhibitory RNA, is sufficiently complementary if it binds to the target sequence under predetermined conditions and inhibits target gene expression. For example, an antisense nucleotide sequence can be designed to specifically hybridize to the replication or transcription regulatory regions of a target gene, or the translation regulatory regions such as translation initiation region and exon/intron junctions, or the coding regions of a target mRNA.

For example, an antisense nucleotide sequence can be designed to specifically hybridize to the replication or transcription regulatory regions of a target gene, or the translation regulatory regions such as translation initiation region and exon/intron junctions, or the coding regions of a target mRNA. In specific embodiments, the shRNA targets a sequence that encodes the N-terminal region of the Sav1 protein, sequence that encodes the middle of the Sav1 protein, or sequence that encodes the C-terminal region of the Sav1 protein.

a. shRNA: Synthesis

As is generally known in the art, commonly used oligonucleotides are oligomers or polymers of ribonucleic acid or deoxyribonucleic acid having a combination of naturally-occurring purine and pyrimidine bases, sugars and covalent linkages between nucleosides including a phosphate group in a phosphodiester linkage. However, it is noted that the term “oligonucleotides” also encompasses various non-naturally occurring mimetics and derivatives, i.e., modified forms, of naturally-occurring oligonucleotides as described below.

shRNA molecules of the disclosure can be prepared by any method known in the art for the synthesis of DNA and RNA molecules. These include techniques for chemically synthesizing oligodeoxy-ribonucleotides and oligo-ribonucleotides well known in the art such as, for example, solid phase phosphoramidite chemical synthesis. Alternatively, RNA molecules can be generated by in vitro and in vivo transcription of DNA sequences encoding the shRNA molecule. Such DNA sequences may be incorporated into a wide variety of vectors that incorporate suitable RNA polymerase promoters such as the T7 or SP6 polymerase promoters. Alternatively, antisense cDNA constructs that synthesize shRNA constitutively or inducibly, depending on the promoter used, can be introduced stably into cell lines.

shRNA molecules can be chemically synthesized using appropriately protected ribonucleoside phosphoramidites and a conventional DNA/RNA synthesizer. Custom shRNA synthesis services are available from commercial vendors such as Ambion (Austin, Tex., USA) and Dharmacon Research (Lafayette, Colo., USA). See, e.g., U.S. Pat. No. 7,410,944.

Various well-known modifications to the DNA molecules can be introduced as a means of increasing intracellular stability and half-life. Possible modifications include, but are not limited to, the addition of flanking sequences of ribo- or deoxy-nucleotides to the 5′ and/or 3′ ends of the molecule or the use of phosphorothioate or 2′ O-methyl rather than phosphodiesterase linkages within the oligodeoxyribonucleotide backbone. An antisense nucleic acid of the invention can be constructed using chemical synthesis or enzymatic ligation reactions using procedures known in the art. An antisense oligonucleotide can be chemically synthesized using naturally-occurring nucleotides or variously modified nucleotides designed to increase the biological stability of the molecules or to increase the physical stability of the duplex formed between the antisense and sense nucleic acids (e.g., phosphorothioate derivatives and acridine substituted nucleotides can be used).

The shRNA molecules of the invention can be various modified equivalents of the structures of any Sav1 shRNA. A “modified equivalent” means a modified form of a particular siRNA molecule having the same target-specificity (i.e., recognizing the same mRNA molecules that complement the unmodified particular siRNA molecule). Thus, a modified equivalent of an unmodified siRNA molecule can have modified ribonucleotides, that is, ribonucleotides that contain a modification in the chemical structure of an unmodified nucleotide base, sugar and/or phosphate (or phosphodiester linkage). See, e.g., U.S. Pat. No. 7,410,944.

Preferably, modified shRNA molecules contain modified backbones or non-natural internucleoside linkages, e.g., modified phosphorous-containing backbones and non-phosphorous backbones such as morpholino backbones; siloxane, sulfide, sulfoxide, sulfone, sulfonate, sulfonamide, and sulfamate backbones; formacetyl and thioformacetyl backbones; alkene-containing backbones; methyleneimino and methylenehydrazino backbones; amide backbones, and the like. See, e.g., U.S. Pat. No. 7,410,944.

Examples of modified phosphorous-containing backbones include, but are not limited to phosphorothioates, phosphorodithioates, chiral phosphorothioates, phosphotriesters, aminoalkylphosphotriesters, alkyl phosphonates, thionoalkylphosphonates, phosphinates, phosphoramidates, thionophosphoramidates, thionoalkylphosphotriesters, and boranophosphates, and various salt forms thereof. See, e.g., U.S. Pat. No. 7,410,944.

Examples of the non-phosphorous containing backbones described above are known in the art, e.g., U.S. Pat. No. 5,677,439, each of which is herein incorporated by reference. See, e.g., U.S. Pat. No. 7,410,944.

Modified forms of shRNA compounds can also contain modified nucleosides (nucleoside analogs), i.e., modified purine or pyrimidine bases, e.g., 5-substituted pyrimidines, 6-azapyrimidines, pyridin-4-one, pyridin-2-one, phenyl, pseudouracil, 2,4,6-trimethoxy benzene, 3-methyl uracil, dihydrouridine, naphthyl, aminophenyl, 5-alkylcytidines (e.g., 5-methylcytidine), 5-alkyluridines (e.g., ribothymidine), 5-halouridine (e.g., 5-bromouridine) or 6-azapyrimidines or 6-alkylpyrimidines (e.g. 6-methyluridine), 2-thiouridine, 4-thiouridine, 5-(carboxyhydroxy methyl)uridine, 5′-carboxymethylaminomethyl-2-thiouridine, 5-carboxymethylaminomethyluridine, 5-methoxyaminomethyl-2-thiouridine, 5-methylaminomethyluridine, 5-methylcarbonylmethyl uridine, 5-methyloxyuridine, 5-methyl-2-thiouridine, 4-acetylcytidine, 3-methylcytidine, propyne, quesosine, wybutosine, wybutoxosine, beta-D-galactosylqueosine, N-2, N-6 and O-substituted purines, inosine, 1-methyladenosine, 1-methylinosine, 2,2-dimethylguanosine, 2-methyladenosine, 2-methylguanosine, N6-methyladenosine, 7-methylguanosine, 2-methylthio-N-6-isopentenyl adenosine, beta-D-mannosylqueosine, uridine-5-oxyacetic acid, 2-thiocytidine, threonine derivatives, and the like. See, e.g., U.S. Pat. No. 7,410,944.

In addition, modified shRNA compounds can also have substituted or modified sugar moieties, e.g., 2′-O-methoxyethyl sugar moieties. See, e.g., U.S. Pat. No. 7,410,944.

b. shRNA: Administration

The present disclosure provides a composition of a polymer or excipient and one or more vectors encoding one or more shRNA molecules. The vector can be formulated into a pharmaceutical composition with suitable carriers and administered to a patient using a suitable route of administration.

In particular, shRNA compounds may be administered systemically, for example, via parenteral administration. They can also be delivered directly to a particular organ or tissue by any suitable localized administration methods such as direct injection into a target tissue.

In particular embodiments, the shRNA molecules are comprised in a vector, including a viral or non-viral vector. In specific embodiments, the vector is non-integrating, although in other embodiments it is integrating. Viral vectors may be lentiviral, adenoviral, adeno-associated viral, and retroviral, for example. Non-viral vectors include plasmids. In specific embodiments, the AAV9 vector (Piras et al., 2013) is employed. Vectors may be delivered to an individual systemically or locally. In certain embodiments, the vectors utilize tissue-specific or cell-specific promoters, such as cardiomyocyte-specific promoters. In specific embodiments, the vectors are delivered by local injection.

One route of administration of shRNA molecules of the disclosure includes direct injection of the vector into heart tissue such as for example, the myocardium.

In general, included in the invention is a vector comprising a polynucleotide sequence, and a promoter operably-linked to an isolated nucleic acid sequence encoding a first segment, a second segment located immediately 3′ of the first segment, and a third segment located immediately 3′ of the second segment, wherein the first and third segments are each less than 30 base pairs in length and each more than 10 base pairs in length, and wherein the sequence of the third segment is the complement of the sequence of the first segment. The second segment, located immediately 3′ of the first segment, encodes a loop structure containing from 4-10 nucleotides (i.e., at least, at most, or exactly 4, 5, 6, 7, 8, 9, 10). The nucleic acid sequence is expressed as an siRNA and functions as a small hairpin RNA molecule (shRNA) targeted against a designated nucleic acid sequence.

More specifically, the present disclosure includes compositions and methods for selectively reducing the expression of the gene product from Sav1. The present invention provides a vector comprising a polynucleotide sequence which comprises a nucleic acid sequence encoding a shRNA targeted against Sav1. The shRNA forms a hairpin structure comprising a duplex structure and a loop structure. The loop structure may contain from 4 to 10 nucleotides, such as 4, 5 or 6 nucleotides. The duplex is less than 30 nucleotides in length, such as from 10 to 27 nucleotides. The shRNA may further comprise an overhang region. Such an overhang may be a 3′ overhang region or a 5′ overhang region. The overhang region may be, for example, at least, at most, or exactly 1, 2, 3, 4, 5, or 6 nucleotides in length.

In one aspect of the invention, multiple vectors each encoding a different shRNA (targeted to a different region of the Sav1 nucleic acid sequence) may be administered simultaneously or consecutively to a patient. An individual vector may encode multiple shRNAs targeted to different areas of the same gene; i.e., comprising two or more of a shRNA comprising SEQ ID NO: 2 and shRNA comprising SEQ ID NO: 3 and a shRNA comprising SEQ ID NO: 4. In another aspect, an individual vector may encode multiple copies of shRNA comprising SEQ ID NO: 2 or multiple copies of shRNA comprising SEQ ID NO: 3 or multiple copies of shRNA comprising SEQ ID NO: 4, in any ratio.

The vector of the disclosure may further comprise a promoter. Examples of promoters include regulatable promoters and constitutive promoters. For example, the promoter may be a CMV or RSV promoter. The vector may further comprise a polyadenylation signal, such as a synthetic minimal polyadenylation signal. Many such promoters are known in the art and are envisioned for use in this invention. In other instances, the promoter may be a tissue specific promoter, such as a cardiac tissue specific promoter.

The vector may further comprise one or more marker genes or reporter genes. Many marker genes and reporter genes are known in the art. The present invention contemplates use of one or more marker genes and/or reporter genes known in the art in the practice of the invention. The marker genes or reporter genes provide a method to track expression of one or more linked genes. The marker genes or reporter genes upon expression within the cell, provide products, usually proteins, detectable by spectroscopic, photochemical, biochemical, immunochemical, chemical, or other physical means. Gene expression products, whether from the gene of interest, marker genes or reporter genes may also be detected by labeling. Labels envisioned for use in the inventions included herein include, but are not limited to, fluorescent dyes, electron-dense reagents, enzymes (for example, as commonly used in an ELISA), biotin, digoxigenin, or haptens and proteins which can be made detectable, e.g., by incorporating a radiolabel into the peptide or used to detect antibodies specifically reactive with the peptide. See, e.g., U.S. Pat. No. 7,419,779.

c. shRNA: Pharmaceutical Compositions

The shRNA encoding nucleic acids of the present disclosure can be formulated in pharmaceutical compositions. The pharmaceutical compositions of the invention comprise a therapeutically effective amount of the vector encoding shRNA. These compositions can comprise, in addition to the vector, a pharmaceutically acceptable excipient, carrier, buffer, stabilizer, or other materials. Such materials should be non-toxic and should not interfere with the efficacy of the active ingredient. The carrier can take a wide variety of forms depending on the form of preparation desired for administration.

When the vectors of the disclosure are prepared for administration, they may be combined with a pharmaceutically acceptable carrier, diluent, or excipient to form a pharmaceutical formulation, or unit dosage form.

For example, the vectors can be formulated in buffer solutions such as phosphate buffered saline solutions, in physiological saline, or in water. Other pharmaceutically acceptable carriers, excipients, stabilizers, and/or preservatives can be included in the formulations.

It will be recognized that the choice of a pharmaceutically acceptable carrier, including a physiologically acceptable compound, depends, for example, on the route of administration of the composition.

Formulations of vectors with cationic lipids can be used to facilitate transfection of the vectors into isolated cells. For example, cationic lipids, such as lipofectin, cationic glycerol derivatives, and polycationic molecules, such as polylysine, can be used. Suitable lipids include, for example, Oligofectamine and Lipofectamine (Life Technologies) which can be used according to the manufacturer's instructions.

The shRNA or a composition comprising it may be administered in a therapeutically effective amount. An “effective amount” or a “therapeutically effective amount” is an amount sufficient to carry out a specifically stated purpose, such as improving heart function, improving systolic function, reducing arrhythmias, and/or promoting capillary formation in heart tissue.

d. shRNA: Gene Therapy

siRNA can also be delivered into mammalian cells, particularly human cells, by a gene therapy approach, using a DNA vector from which siRNA compounds in, e.g., small hairpin form (shRNA), can be transcribed directly. Recent studies have demonstrated that while double-stranded siRNAs are very effective at mediating RNAi, short, single-stranded, hairpin-shaped RNAs can also mediate RNAi, presumably because they fold into intramolecular duplexes that are processed into double-stranded siRNAs by cellular enzymes. This discovery has significant and far-reaching implications, since the production of such shRNAs can be readily achieved in vivo by transfecting cells or tissues with DNA vectors bearing short inverted repeats separated by a small number (e.g., 3, 4, 5, 6, 7, 8, 9) of nucleotides that direct the transcription of such small hairpin RNAs. Additionally, if mechanisms are included to direct the integration of the vector or a vector segment into the host-cell genome, or to ensure the stability of the transcription vector, the RNAi caused by the encoded shRNAs can be made stable and heritable.

Gene therapy is carried out according to generally accepted methods as are known in the art. See, e.g., U.S. Pat. Nos. 5,837,492 and 5,800,998 and references cited therein. Vectors in the context of gene therapy are meant to include those polynucleotide sequences containing sequences sufficient to express a polynucleotide encoded therein. If the polynucleotide encodes an shRNA, expression will produce the antisense polynucleotide sequence. Thus, in this context, expression does not require that a protein product be synthesized. In addition to the shRNA encoded in the vector, the vector also contains a promoter functional in eukaryotic cells. The shRNA sequence is under control of this promoter. Suitable eukaryotic promoters include those described elsewhere herein and as are known in the art. The expression vector may also include sequences, such as selectable markers, reporter genes and other regulatory sequences conventionally used.

Accordingly, the amount of shRNA generated in situ is regulated by controlling such factors as the nature of the promoter used to direct transcription of the nucleic acid sequence, (i.e., whether the promoter is constitutive or regulatable, strong or weak) and the number of copies of the nucleic acid sequence encoding a shRNA sequence that are in the cell.

For expression of Sav1 shRNA, a promoter is operatively linked to a shRNA sequence. As used herein, the term “promoter” refers to a DNA sequence that regulates expression of the target gene sequence being operatively linked to the promoter sequence in a certain host cell. The term “operatively linked” or “operably linked” means that one nucleic acid fragment is linked to another nucleic acid fragment so that the function or expression thereof is affected by the other nucleic acid fragment. The expression cassette of the present invention may further comprise various expression regulatory sequences such as an optional operator sequence for controlling transcription, a sequence encoding a suitable mRNA ribosome-binding site, and sequences controlling the termination of transcription and translation. The promoter used in the present invention may be a constitutive promoter that constitutively induces the expression of a target gene, or an inducible promoter that induces the expression of a target gene at a given position and time point. Specific examples of the promoter may include U6 promoter, CMV (cytomegalovirus) promoter, SV40 promoter, CAG promoter (Hitoshi Niwa et al., Gene, 108:193-199, 1991; and Monahan et al., Gene Therapy, 7:24-30, 2000), CaMV 35S promoter (Odell et al., Nature 313:810-812, 1985), Rsyn7 promoter (U.S. patent application Ser. No. 08/991,601), ubiquitin promoter (Christensen et al., Plant Mol. Biol. 12:619-632, 1989), ALS promoter (U.S. patent application Ser. No. 08/409,297) and the like. Also usable promoters are disclosed in U.S. Pat. Nos. 5,608,149, 5,608,144, 5,604,121, 5,569,597, 5,466,785, 5,399,680, 5,268,463, 5,608,142, etc.

The recombinant vector of the present disclosure may be introduced into an isolated host cell, using a conventional method known in the art. The host cell may be employed for manipulation of the vector or as a means to transfer the vector to an individual. Preferably, intracellular incorporation of the vector into the host cell may be carried out by a conventional method known in the art, such as calcium chloride, microprojectile bombardment, electroporation, PEG-mediated fusion, microinjection, liposome-mediated method, and the like.

Examples of the isolated host cell that can be utilized in the present disclosure may include, but are not limited to, prokaryotic cells such as Escherichia coli, Bacillus subtilis, Streptomyces, Pseudomonas, Proteus mirabilis, and Staphylococcus, lower eukaryotic cells such as fungi (e.g., Aspergillus), yeast (e.g., Pichia pastoris), Saccharomyces cerevisiae, Schizosaccharomyces, and Neurospora crassa, and higher eukaryotic cells such as insect cells, plant cells, mammalian cells. Preferably, the host cell may be human cells.

Meanwhile, standard recombinant DNA and molecular cloning techniques used in the present disclosure are well known in the art and can be found in the following literature: Sambrook, J., Fritsch, E. F. and Maniatis, T., Molecular Cloning: A Laboratory Manual, 2nd ed., Cold Spring Harbor Laboratory: Cold Spring Harbor, N.Y. (1989); Silhavy, T. J., Bennan, M. L. and Enquist, L. W., Experiments with Gene Fusions, Cold Spring Harbor Laboratory: Cold Spring Harbor, N.Y. (1984); and Ausubel, F. M. et al., Current Protocols in Molecular Biology, published by Greene Publishing Assoc. and Wiley-Interscience (1987).

### EXAMPLES

The following examples are included to demonstrate certain non-limiting aspects of the invention. It should be appreciated by those of skill in the art that the techniques disclosed in the examples that follow represent techniques discovered by the inventors to function well in the practice of the invention. However, those of skill in the art should, in light of the present disclosure, appreciate that many changes can be made in the specific embodiments that are disclosed and still obtain a like or similar result without departing from the spirit and scope of the invention

### Example 1

### Viral Delivery of Sav SHRNA Improves Systolic Function in a Pig Model of Myocardial Infarction

Adeno-associated virus 9 (AAV9)-based gene therapy was used to locally knock down the Hippo pathway gene Say in border zone (BZ) cardiomyocytes (CM) in a pig model of ischemia-reperfusion-induced myocardial infarction (MI). Two weeks after MI, when pigs had left ventricular (LV) systolic dysfunction, AAV9-Sav-shRNA or control virus was administered directly to BZ CMs via catheter-mediated subendocardial injection. Three months after injection, hearts treated with a high dose of AAV9-Sav-shRNA had a 14.3% improvement in ejection fraction (a measure of LV systolic function), evidence of CM division, and reduced scar sizes compared with controls. None of the animals died during the three-month follow-up period or demonstrated uncontrolled proliferation or de-maturation of cardiomyocytes.

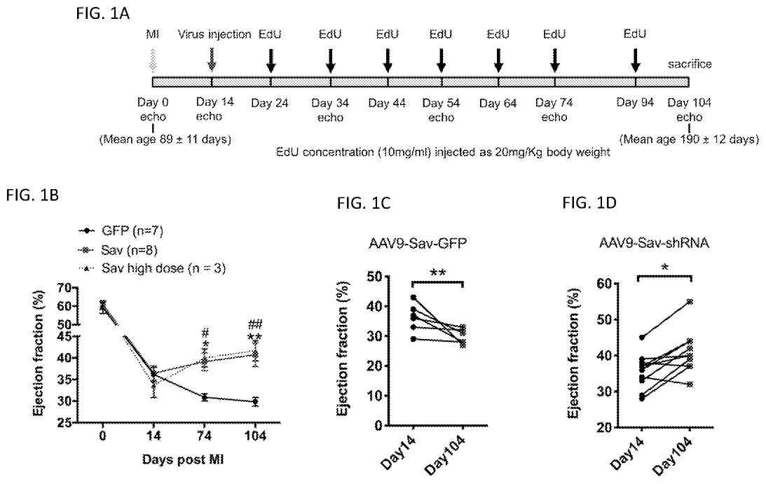

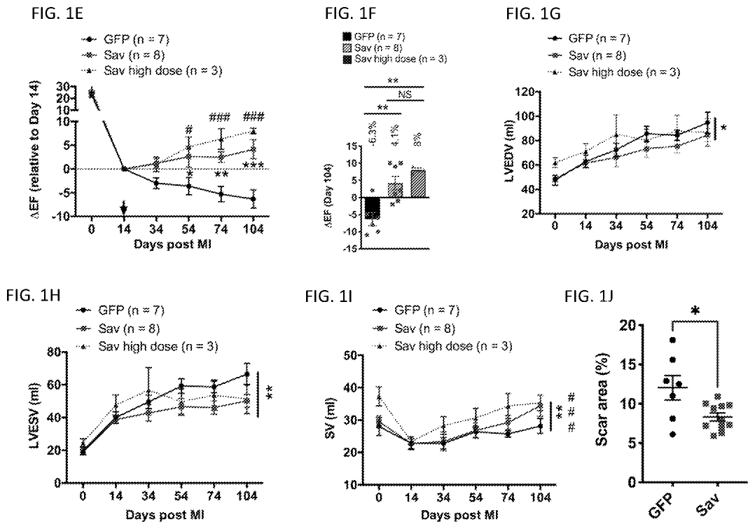

In greater detail, to determine whether AAV9-Sav-shRNA promotes heart regeneration after MI, an angioplasty balloon was used to induce MI via ischemia/reperfusion (I/R) in ˜3-month old pig hearts by transiently occluding the left anterior descending (LAD) artery for 90 minutes, followed by reperfusion (FIG. 1A). Two weeks after MI, echocardiography (echo) revealed a reduction in left ventricular ejection fraction (EF) to below 40% in both groups of pigs (AAV9-GFP, EF: 36.2±2.0% [mean±SEM]; AAV9-Sav-shRNA, EF: 36.5±1.7% [mean±SEM]) (FIG. 1B; Table 1), indicating significantly decreased heart function.

TABLE 1
Echocardiographic Results Showing Decreased
Heart Function 14 Days Post-MI

| P-1946 | EF | LVEDV | LVESV | SV |
|---|---|---|---|---|
| Day 0 | 60% | 59 mL | 23 mL | 36 mL |
| Day 14 | 30% | 65 mL | 46 mL | 23 mL |

EF: ejection fraction;
LVEDV: left ventricular end-diastolic volume;
LVESV: left ventricular end-systolic volume;
SV: systolic volume

Moreover, transmural scars in the anterior wall were prominent, indicating that MI was successfully induced by the I/R procedure. AAV9-GFP or AAV9-Sav-shRNA was injected into border zone (BZ) myocardium using NOGA mapping to localize the injection sites, and echo was performed at 20, 40, 60, and 90 days after virus injections to longitudinally monitor heart function in each pig (FIG. 1A).

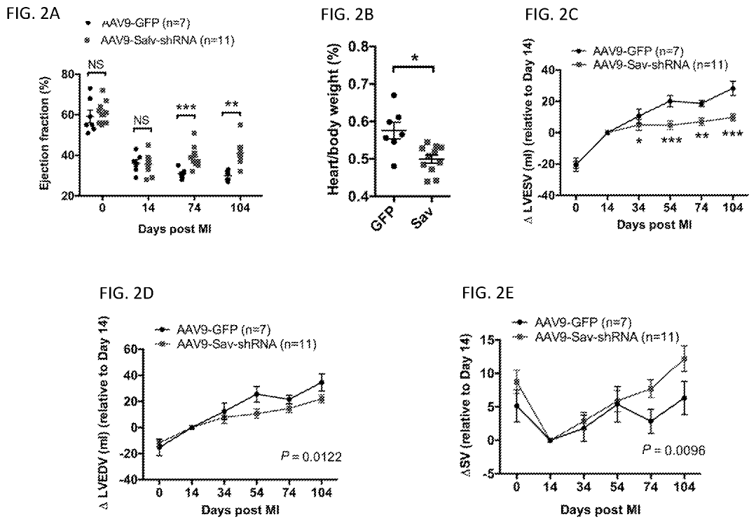

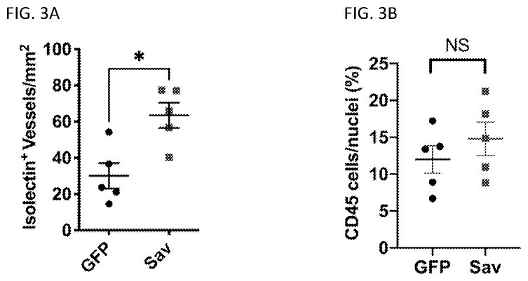

Notably, the longitudinal analyses of AAV9-GFP-injected pig hearts revealed that LV function steadily worsened, consistent with progressive pathologic remodeling. In contrast, in AAV9-Sav-shRNA-injected hearts, LV function progressively improved over time (FIGS. 1B-1F; FIG. 2A). In AAV9-GFP-injected hearts, EF dropped an additional 6.3±1.9% during the three-month follow-up period (FIGS. 1C, 1E, and 1F), whereas in the low-dose AAV9-Sav-shRNA-injected hearts, EF increased by 4.1±2% (1×10¹³ viral genome copies/experimental group) (FIGS. 1C, 1D, and 1F). Three pigs, administered a high-dose of AAV9-Sav-shRNA (4×10¹³ viral genome copies), exhibited a robust improvement in EF (8.0±0.5%) at day 104 compared with day 14 (FIGS. 1E and 1F). The difference in EF between high and low viral doses failed to reach statistical significance, most likely because of the limited number of pigs (FIGS. 1E and 1F). AAV9-GFP control-treated pigs exhibited an increase in absolute heart weights and heart weight-to-body weight (HW/BW) ratios compared with AAV9-Sav-shRNA-treated pigs, suggesting that control pigs underwent cardiac hypertrophy (FIG. 2B).

Importantly, AAV9-Sav-shRNA treatment improved LV systolic function over time with only a marginal effect on diastolic function, indicating that improved EF was primarily due to restored LV systolic function (FIGS. 1G, 1H; FIGS. 2C, 2D). Consistent with this, compared with AAV9-GFP-injected hearts, AAV9-Sav-shRNA-injected hearts showed improved stroke volume (FIG. 1I, FIG. 2E). Three months after virus injections, all pigs were euthanized, and hearts were carefully analyzed. Transmural scars were observed in all hearts and were notably smaller in AAV9-Sav-shRNA-injected hearts than in control hearts (FIG. 1J, Table 2). After sectioning ventricles into seven slices, the infarct area was measured in each slice. Significantly reduced scarring was observed in AAV9-Sav-shRNA-injected hearts compared with control hearts (FIG. 1J). Together, these data indicate that that AAV9-Sav-shRNA improves LV systolic heart function and reduces scar formation in a pig model of MI.

TABLE 2
Evaluation of Infarct Size

| Animal ID | MI Age (days) | Infarct Size % (MI/LV) |
|---|---|---|
| P-1894 | 75 | 12.5 |
| P-1895 | 75 | 10.9 |
| P-1896 | 104 | 8.1 |
| P-1897 | 104 | 7.1 |
| P-1903 | 104 | 6.3 |
| P-1917 | 104 | 9.8 |
| P-1918 | 104 | 18.1 |
| P-1964 | 15 | 24.1 |
| P-1937 | 104 | 9.7 |
| P-1947 | 104 | 11 |
| P-1949 | 104 | 15.6 |
| P-1950 | 104 | 12.9 |
| P-1953 | 104 | 7.3 |
| P-1956 | 104 | 7.9 |
| P-1960 | 104 | 7.2 |
| P-2019 | 104 | 10 |
| P-2020 | 104 | 6.1 |
| P-2022 | 104 | 5.9 |

MI: myocardial infarction;
LV: left ventricle

Notably, blood test results revealed normal white and red blood cell counts, as well as normal liver and kidney function, 45 days after viral injection. Liver and lung tissues were closely examined 3 months after injection, and no tumors were observed, indicating that localized infection of AAV9-Sav-shRNA is safely tolerated.

### Example 2

### Viral Delivery of Sav SHRNA Promotes Vascularity in Pig Hearts after Myocardial Infarction

AAV9-Sav-shRNA-treated hearts displayed increased capillary density and reduced CM ploidy. In particular, to determine whether AAV9-Sav-shRNA infection promotes vascularity in pig hearts after MI, IF staining was performed for the endothelial marker isolectin B4. Tiled image analysis revealed more capillary formation in low-dose AAV9-Sav-shRNA-injected hearts than in control hearts. Endothelial cells were observed in the infarct tissue of pig hearts at 90 days after virus injection. Quantification is shown in FIG. 3A.

Inflammatory responses were investigated using IF to determine the number of CD45-positive leukocytes in the BZ of hearts after MI. IF staining for CD45 revealed no differences in the number of leukocytes in the BZ of AAV9-GFP-injected hearts versus control hearts, most likely because of waning of the immune response three months after MI. Leukocytes were observed in the infarcted tissue of pig hearts at 90 days after virus injection. Quantification is shown in FIG. 3B.

These findings indicate that AAV9-Sav-shRNA infection promotes persistent capillary formation in pig hearts after MI.

### Example 3

### Material and Methods for Examples 1 and 2

AAV9 viral packaging. Viral vectors were used as previously described (Leach et al., 2017). The construct containing triple shRNAs against Say with flanking miR30 sequences was cloned into the pENN.AAV.cTNT, p1967-Q vector downstream of GFP (AAV9-Sav-shRNA). Empty vector encoding GFP was used as the control (AAV9-GFP). Both vectors were packaged into the muscle-trophic serotype AAV9 by the Intellectual and Developmental Disabilities Research Center Neuroconnectivity Core at Baylor College of Medicine. After titering, viruses were aliquoted (1×10¹³ viral genome particles per tube) and immediately frozen and placed at −80° C. for long-term storage. Before each injection, each aliquot was diluted in saline to make a 3 mL injection solution.

Pigs. Animals were handled and maintained in accordance with the requirements of the Laboratory Animal Welfare Act (P.L. 89-544) and its 1970 (P.L. 91-579), 1976 (P.L. 94-279), and 1985 (P.L. 99-198) amendments. Pigs were fed with a commercial diet and fresh clean water in a quantity sufficient to maintain body weight and permit reasonable weight gain in growing animals. Species-appropriate treats were given at the discretion of the attending veterinarian or designee.

When animals underwent surgery, they were given appropriate analgesic and antibiotic medications. Buprenorphine (0.05-0.1 mg/kg), administered intramuscularly or by mouth, was administered every 6 to 12 hours or as deemed necessary by the attending veterinarian. Flunixin meglumine (1.1-2.2 mg/kg) was administered intravenously, intramuscularly, or by mouth every 6 to 8 hours as needed. Naxcel (3-5 mg/kg) or the equivalent was administered intramuscularly once per day for 24 hours starting the day after the procedure. After Naxcel was discontinued, Baytril (enrofloxacin; 2.5-5.0 mg/kg) was administered by mouth twice per day for up to 7 days. Baytril (enrofloxacin; injectable solution, 7.5 mg/kg of body weight (3.4 mL/100 lb)) was administered intramuscularly or subcutaneously (behind the ear). Continued use of antibiotics and analgesics was based on post-procedural monitoring unless otherwise specified. Nitroglycerin paste was applied topically after the MI procedure, followed by aspirin (162.5 mg (½ of a standard 325 mg tablet)) once per day by mouth, and ranitidine (Zantac; 150 mg) 1-3 times per day by mouth. Finally, pantoprazole (40 mg) was administered once per day by mouth for gastroprotection.

EMM and Transendocardial Injections. Endocardial electromechanical mapping (EMM) was performed as previously described (Vale et al., 2000). Briefly, pigs were maintained on general anesthesia and were appropriately prepped and draped in a manner to allow sterile surgical access to the inguinal area. Femoral access was achieved by percutaneous penetration. An introducer sheath was positioned in the artery, and a MyoStar™ injection catheter (Biosense Webster, Diamond Bar, CA, USA) was used to deliver AAV-Sav-shRNA or AAV-GFP. The catheter was advanced into the left ventricle to first create an electromechanical endocardial map. After the completion of point acquisition, the map was processed by using a moderate filter and a manual filter to exclude internal points, points taken outside the LV cavity (e.g., atrial or aortic points), points acquired during premature ventricular contractions, or points that did not fulfill the standard quality criteria (e.g., loop stability >6 mm, location stability >4 mm, cycle length variation >10%). After EMM was completed, the MyoStar™ injection catheter was primed with 0.1 mL of AAV-Sav-shRNA or AAV-GFP to fill the dead space before the start of the injection procedure. The perpendicular positioning of the catheter into the LV wall and the presence of a premature ventricular contraction upon extending the needle into the myocardium ensured proper delivery into the myocardium. For the pig model of MI, the area of infarction was characterized with the NOGA system according to the presence of a low unipolar voltage (i.e., less than 4 mV). BZs, identified by EMM, are typically 5- to 10-mm regions adjacent to scar (Bolli et al., 2018). 10 to 15 transendocardial injections were performed to the BZ of the infarct (unipolar voltage value between 4-7 mV, 0.2 mL per injection). The catheter was then removed and the artery was closed with Angio-seal (Terumo). Pigs were then transferred to the intensive care unit for monitoring.

Acute MI Induced by IR. A 6 F sheet was inserted percutaneously in the common femoral artery. A 5 F coronary guiding catheter was advanced through the aorta, and it was selectively engaged into the left main coronary ostium. Subsequently, an angioplasty balloon (chosen to match the LAD diameter) was positioned into the LAD between the first and second diagonal branch over a floppy 0.014-inch guidewire. The balloon was inflated at nominal pressure, and complete coronary occlusion was documented by the absence of the distal flow of contrast. After 90 minutes, the balloon was deflated, and passive reperfusion to the distal coronary bed was allowed. Patency of the distal circulation was documented by injecting contrast. Ventricular arrhythmias were prevented and treated with lidocaine (1.5 mg/kg boluses followed by continuous infusion), amiodarone (5 mg/kg boluses followed by continuous infusion), and electrical cardioversion, if necessary. During the coronary occlusion period, activated clotting time was periodically measured, and heparin boluses were repeated as needed to keep the ACT range between 250 and 350 seconds. After the procedure was completed, pigs were transferred to the intensive care unit for monitoring.

Two-Dimensional Echocardiography. Under general anesthesia, pigs were placed in the left lateral recumbent position. Oxygen saturation and 12-lead electrocardiograms were monitored. A 3.5 MHz phased-array transducer with a Vivid 7 ultrasound machine (GE Medical Systems, Milwaukee, WI, USA) was used to obtain images, including the parasternal long-axis view, parasternal short-axis view, and apex view. The LV ejection fraction, LV end-systolic volume, and LV end-diastolic volume were calculated by using the bi-plane Simpson method according to the guidelines of the American Society of Echocardiography (Lang et al., 2015). A blinded independent operator performed all analyses.

Pathologic report summary. Hearts and other organs (e.g., lungs, liver, kidneys) were collected from eighteen domestic pigs that underwent MI and reperfusion 2 weeks before receiving transendocardial injections of AAV9-Sav-shRNA or AAV9-GFP virus. A total of 27 animals were included: 7 animals were sacrificed at 45 days after MI, 2 animals were sacrificed at 74 days after MI, and 18 animals were sacrificed at 104 days after MI. Pigs with at least 30% EF reduction (relative to baseline) at 2 weeks after MI were used for further experiments (Koudstaal et al., 2014). Two animals, P-1912 and P-1965, were excluded due to insufficient MI induction. For P-1912, EF was 61% before MI and 44% after MI (a 28% reduction relative to baseline). For P-1965, EF was 54% before MI and 38% after MI (a 29% reduction relative to baseline).

One animal, P-1946, died shortly after receiving transendocardial injections. Examination of the heart revealed a large healing MI in the anterior/anteroseptal LV walls, from the apical to mid ventricular level. Additionally, NOGA-injected segments showed two to three small foci of myocardial hemorrhage near the infarct border. These foci were suggestive of acute injection sites and correlated with sites in the respective two-dimensional NOGA map.

Hearts from all other pigs showed gross and microscopic findings of focal replacement fibrosis that were compatible with a healed MI of the LAD territory. All infarcted hearts showed typical LV remodeling, with marked wall thinning in the involved territory. In a few of the pigs, smaller linear or patchy areas of fibrosis away from ischemic scar were considered procedure-related and were suggestive of healed injection sites or partial needle tracks.

Additionally, small foci of interstitial or perivascular chronic inflammatory infiltrates were observed in 14 of the 18 pigs that were sacrificed at 74 or 104 days after MI. In most of these cases (11 of 14 affected), this finding was either mild or minimal and was occasionally associated with a single or small group of necrotic or vacuolated CMs. However, in three animals, these areas were somewhat more extensive and diffuse (P-1895, P-1918), or showed greater chronicity and myocyte loss with associated interstitial and/or replacement fibrosis (P-1917). The infiltrated areas were present mostly in NOGA-injected segments.

Gross findings in other organs were few and considered incidental or secondary to procedural manipulation. Lungs, liver, and kidneys collected at the time of sacrifice appeared mostly normal upon gross inspection. The lungs had various amounts of dark red discoloration, typically in the dorsal caudal aspect, that were generally attributed to atelectasis due to dorsal recumbency, general anesthesia, and euthanasia. The kidneys had few incidental congenital findings consisting of severe renal hypoplasia (P-1864) and renal cysts (P-1903, P-1947). The livers appeared unremarkable, with only one animal (P-1937) having thinning of the gallbladder wall.

A subset of pigs was selected for a limited microscopic examination of lung and liver tissues. The subset included nine animals: seven study pigs (P-1937, P-1947, P-1949, P-1950, P-1953, P-1956, and P-1960) mixed from both study arms, and an untreated control pig (P-1989). Histologic sections from the middle regions of the respective organ were randomly selected and stained with hematoxylin and eosin (H&E). In all pigs except for one, the lungs showed no significant findings other than some areas of atelectasis that are commonly attributed to the various procedures performed before euthanasia (general anesthesia, dorsal recumbency). One pig, P-1950, had focal interstitial pneumonia in its middle lobes, most likely related to the aspiration of foreign material. No significant findings were observed in the liver, except for minimal focal inflammatory cell aggregates in one animal, P-1960. These findings are considered incidental.

Heart Sample Processing. At the time of euthanization, hearts were excised, weighed, and photographed. After gross evaluation of the exterior, a fresh small sample was taken for cryosectioning and subsequent molecular analysis. Hearts were then perfusion-fixed with 10% neutral-buffered formalin retrograde from the ascending aorta at approximately 100 mm Hg pressure for 20 minutes. After perfusion-fixation, hearts were sliced according to a segmentation protocol designed for the sampling of hearts that underwent transendocardial injection with biologic agents (Vela et al., 2015). Heart slices and base were then immersed in 10% neutral-buffered formalin for 24 to 48 hours. Segments that had received the highest number of injections (as per the respective two-dimensional EMM (NOGA) maps) were selected for analysis. Those segments were additionally sliced into four to five 2 mm thick levels and embedded in different paraffin blocks (usually 40-50 blocks per heart). One to three additional samples from remote noninjected segments were also collected. All samples were processed for paraffin embedding, cut at 5 micron thickness, and stained with H&E.

Infarct Size Calculation. Ventricles were sectioned from apex to base into 7 to 8 transverse slices. Each slice was photographed on its apical and basal face. Image Pro software was used to analyze the digital images. LV and infarct areas were manually traced with ImagePro software from the digital photographs of the apical and basal faces of each slice. For each slice face, the infarct was calculated as a percentage of the LV. The results of both faces were then averaged and multiplied by the slice weight to calculate the size of the infarct. The sum of the infarct sizes from all slices was then divided by the sum of the LV weight from all slices and expressed as a percentage (Jones et al., 2015).

IF Staining. Processed frozen and paraffin sections were used for downstream analyses. For frozen sections, after fixation, hearts were dehydrated in 15% and 30% sucrose gradients and then embedded into optimal cutting temperature compound (Cat #25608-930, VWR International, Radnor, PA, USA) for sectioning. Slides were sectioned at 10 m intervals for IF staining. For paraffin sections, samples were deparaffinized and rehydrated, treated with 3% H₂O₂ in EtOH, treated with antigen retrieval solution (Vector Laboratories, Inc., Burlingame, CA, USA), blocked with 10% donkey serum in phosphate-buffered saline (PBS), and then incubated with primary antibodies. Antibodies were rabbit anti-CD45 (Abeam ab10559) and isolectin B4 (FL-1201, Vector Laboratories).

EdU Incorporation Assay. EdU incorporation was detected by using the Click-iT™ EdU Imaging Kit (Life Technologies, Carlsbad, CA, USA). Imaging of tissue slides was performed with a Leica TCS SP5 confocal microscope, and images were processed by using Leica LAS AF software (Leica Microsystems, Wetzlar, Germany).

Tile Imaging Analysis and Machine Learning. After IF staining, tile images were captured by using a Zeiss LSM 880 with an Airyscan FAST Confocal Microscope at the Optical Imaging & Vital Microscopy Core (OiVM) of Baylor College of Medicine. Each tile image includes at least 8×8=64 scan field at 10× objective-scanned images with 20% overlap for stitching (20-30 mm² per image). Images were then analyzed by Fiji (Schindelin et al., 2012) for machine learning. Pixel-based segmentation of images was produced with the plugin of Trainable Weka Segmentation (Arganda-Carreras et al., 2017), the binary classifications whose area larger than 300 pixels were added to regions of interest (ROIs) in Fiji. DAPI staining in an area larger than 100 pixels was counted as nuclei, and the overlap between nuclei and ROIs was detected by Speckle Inspector and counted as one cell (Brocher, 2014). Cells in which nuclei were attached to the cell boundary (non-CMs) were filtered out using intensity analysis on the updated ROI. Finally, CMs that overlapped with EdU staining were counted as EdU-positive CMs.

Statistics. Throughout the study, all analyses were performed in a double-blinded manner. Data are presented as the mean±standard error of the mean. Quantitative data for two groups were evaluated for significance by using the Mann-Whitney U-test. For comparisons among multiple groups, we used two-way analysis of variance with Bonferroni's pairwise post-hoc test. A p-value of less than 0.05 was considered significant for all analyses. Significant differences between experimental groups were denoted as *P<0.05, **P<0.01, or ***P<0.001.

Each animal received treatment as multiple discrete injections into the left ventricle of the heart. Injection sites were mapped/tracked using a physical (NOGA) map and a GFP reporter for cells infected by the virus. Each injection site serves as an island of infected cells surrounded by noninfected cells in between. Histology segments were collected from injection locations for analysis (not an equal number from each animal due to limitations of sample availability and histology preparations). Each segment has a measurement value. Because each injection site can be defined and cells that are infected with the virus can be tracked, each tile image/segment was categorized, rather than each pig, as the replicate. Data was displayed in a nested graph format, while keeping individual tile image/segments as replicates in the nested figure, and ANOVA with post-testing was used to assess the effect of AAV9-Sav-shRNA treatment on the cells' ability to enter the cell cycle. These analyses were performed using Graphpad Prism software. For the ANOVA analysis, the assumption of independence was tested by using the chi-square test, normality was tested by using a normal QQ plot, and equality of variance was tested Brown-Forsythe test or Bartlett's test. If the tests indicated that the homogeneity of variances assumption is invalid, the non-parametric Kruskal-Wallis ANOVA test was used.

### Example 4

### Gene Therapy Knockdown of Hippo Signaling Resolves Arrhythmic Events in Pigs after Myocardial Infarction

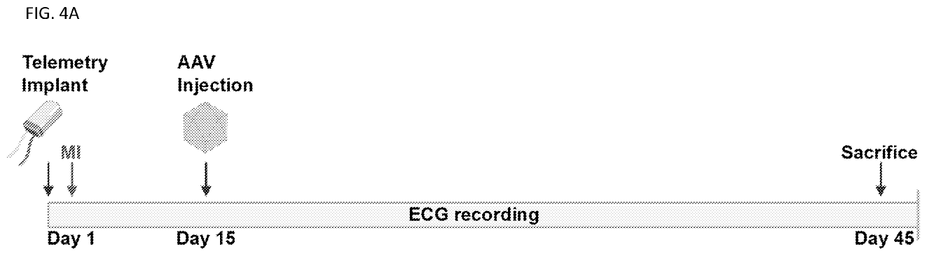

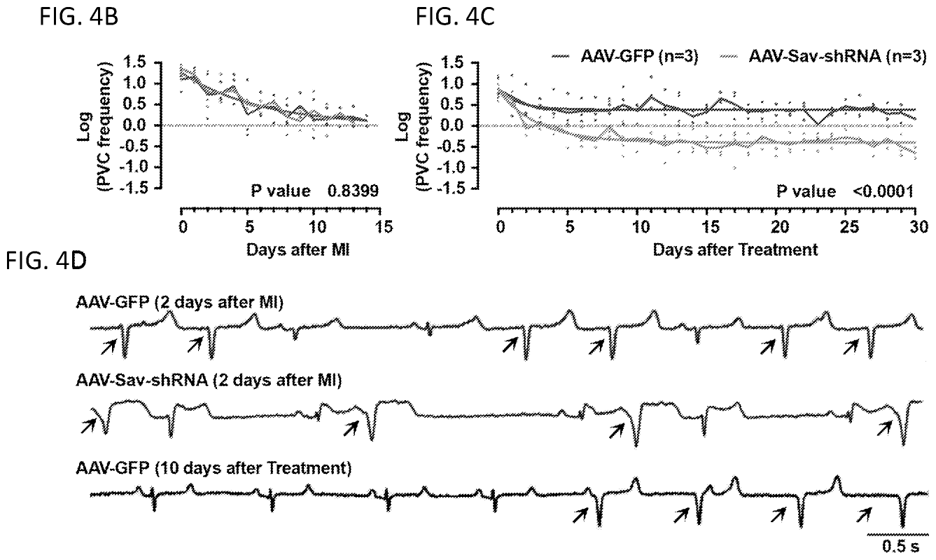

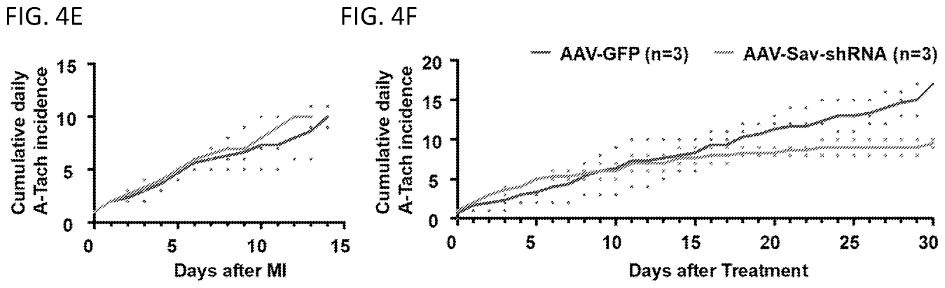

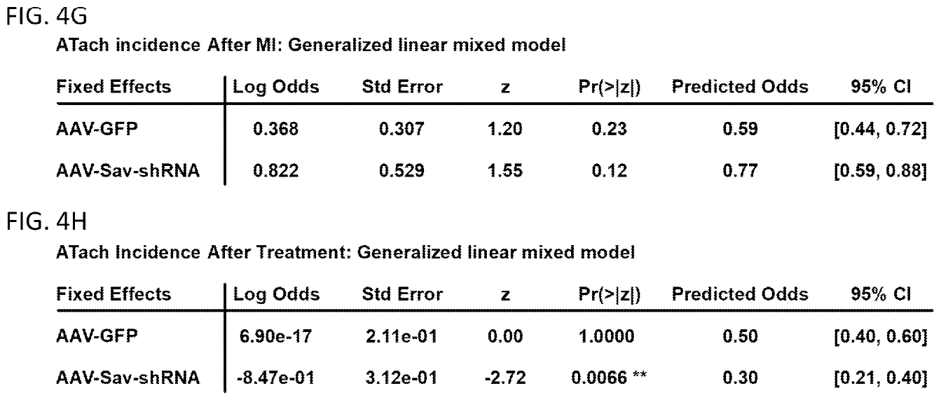

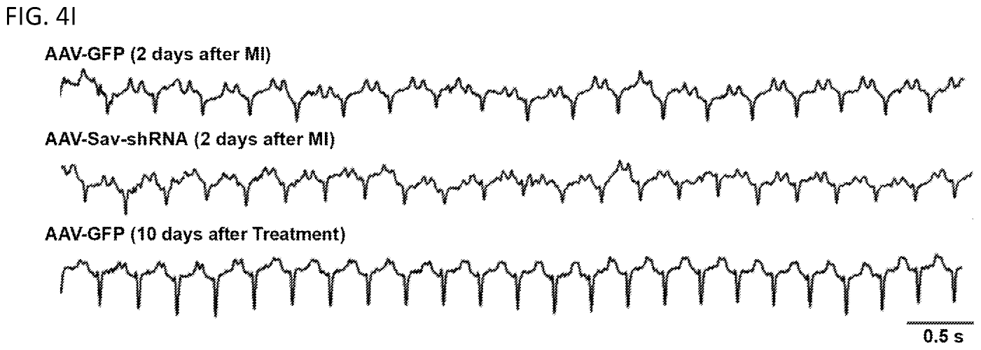

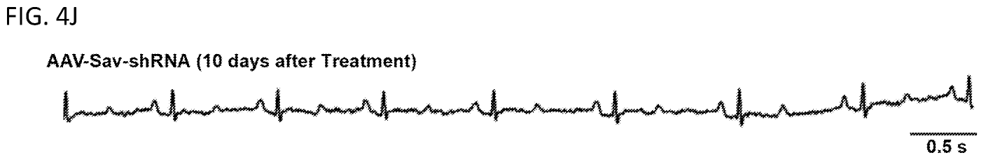

After myocardial infarction, the heart is incapable of meaningful regeneration, and ischemic/reperfusion injuries lead to the loss of millions of cardiomyocytes and initiate a pathologic fibrotic remodeling while progressively impairing contractile function. Genetic manipulation of a variety of developmental and cell cycle pathways has demonstrated that inducing cardiomyocyte renewal has the potential to restore muscle tissue and improve cardiac function. However, uncontrolled cardiomyocyte proliferation has been associated with detrimental effects on electrical conduction leading to sudden and deadly arrhythmogenic events in large animal models of myocardial infarction (Riching et al., 2021; Gabisonia et al., 2019). Reports of arrhythmogenic events prompted verification of cardiac electrical conduction in a pig model of myocardial infarction using an AAV-based shRNA gene therapy (FIG. 4A).

Before surgically induced MI, a subcutaneous telemetry device (easyTEL+L-EEEETA manufactured by emka Technologies) was implanted subcutaneously to monitor cardiac rhythm. Leads were extended to the 4 limbs through subcutaneous tunnels to obtain 6 lead ECG recordings. Then, a 10-15 min continuous ECG recording was saved for each pig before MI. To induce MI, an angioplasty balloon was positioned into the left anterior descending artery between the first and second diagonal branch and inflated to block the blood flow. Complete coronary occlusion proceeded for 90 minutes and was documented by the absence of the distal flow of contrast. After a 14-day recovery, viral vector was delivered, and ECG recording was continued for another 30 days. The total ECG recording period was approximately 45 days until the animals were euthanized (FIG. 4A). From each pig, all 24 hr ECG recording segments for the full 45-day duration were reviewed for electric and mechanical interferences as well as artifacts. ECG segments recorded during the night had the least interference, therefore, analysis was focused on premature ventricular complexes (PVC, FIGS. 4B-4D) and atrial tachycardia (A-Tach, FIGS. 4E-4J) in the ECG segments recorded between midnight and 6 am.

To aid in the identification and quantification of ventricular and atrial arrhythmias, semi-automated analysis software (ecgAUTO V3.5.5.27) was used. A library of different waveforms was stored for each 6-hour ECG segment, including normal complex waveforms. As expected, a variety of arrhythmia phenotypes could be identified in the period immediately following the MI procedure. Ventricular arrhythmias were found during the duration of the study period (45 days), and incidence tapered over time (FIGS. 4B-4D). Within 3 days of MI, there were multiple severe ventricular arrhythmia phenotypes including intraventricular conduction delay, premature ventricular complexes ([PVC], FIGS. 4B, 4D), bigeminal PVCs, PVC couplets, and non-sustained ventricular tachycardia. One example of a focus was on potentially lethal ventricular arrhythmia, particularly premature ventricular complexes. The PVC frequency per ECG complexes was determined daily in each midnight-to-6 am period for the duration of the experimental timeline. As expected, a mixed effects analysis of PVC frequency in the period after MI and before treatment indicates a significant decrease in the fixed effect of time (P=0.0065) with no difference between treatment groups (P=0.6400, FIG. 4B). Additionally, a single nonlinear curve fit describes both datasets (P=0.8399). After treatment, PVC-frequency decreased over time (P=<0.0001) as well as with AAV-Sav-shRNA treatment (P=0.025), with different curves to describe nonlinear fit between the treatment groups (P=<0.0001, FIG. 4C). At 10 days after treatment, PVCs were identified in AAV-GFP (FIG. 4D), whereas PVCs were minimal in the animals treated with AAV-Sav-shRNA (FIG. 4J).

Similarly, there were multiple atrial rhythm abnormalities including premature atrial complexes, atrial flutter, atrial fibrillation, and atrial tachycardia (FIGS. 4E-4I). All atrial arrhythmias were paroxysmal and quickly subsided, especially in the early stages after MI (FIG. 4I). Here, the focus was on atrial tachycardia, and the cumulative number of daily tachycardia events observable within the midnight-to-6 am period for the duration of the experiment were counted (FIGS. 4E, 4F). Before treatment, there was no discernable difference in the incidence of atrial tachycardia between the AVV-GFP and AAV-Sav-shRNA groups (FIG. 4E). Approximately 2 weeks after treatment with AAV-Sav-shRNA, atrial tachycardia events were no longer detected, and thus, the cumulative incidence plateaued (FIG. 4F). Atrial tachycardia incidence was analyzed with a generalized linear mixed model and generated predicted odds and 95% confidence intervals before AAV injection (AAV-GFP, 0.59 [0.44, 0.72]; AAV-Sav-shRNA, 0.77 [0.59, 0.88], FIG. 4G) and after AAV injection (AAV-GFP, 0.50 [0.40, 0.60]; AAV-Sav-shRNA, 0.30 [0.21, 0.40], FIG. 4H). The odds of observing atrial tachycardia in an animal treated with AAV-Sav-shRNA drops significantly (P(>|z|)=0.0066, FIG. 4H). Given that most cardiomyocyte proliferation occurs in the early stage of treatment (30 days after viral injection), the drop in PVC frequency and atrial tachycardia incidence along with the observation that none of the animals died during the three month follow-up period or demonstrated uncontrolled proliferation or de-maturation of cardiomyocytes (see Example 1) indicates that the adult cardiomyocyte renewal approach does not inherently induce arrhythmogenesis. Finally, since post infarct arrhythmia, particularly refractory ventricular arrhythmias, contribute significantly to death of patients surviving myocardial infarction (Bhar-Amato et al., 2017), the gene therapy used for cardiomyocyte regeneration also has therapeutic values in reducing frequencies of potentially fatal ventricular arrhythmias after myocardial infarction.

### REFERENCES

Arganda-Carreras, I. et al. Trainable Weka Segmentation: a machine learning tool for microscopy pixel classification. Bioinformatics 33, 2424-2426, doi:10.1093/bioinformatics/btx180 (2017).
Bhar-Amato J, Davies W, Agarwal S. Ventricular Arrhythmia after Acute Myocardial Infarction: ‘The Perfect Storm’. Arrhythm Electrophysiol Rev. 2017 August; 6(3):134-139. doi: 10.15420/aer.2017.24.1. PMID: 29018522; PMCID: PMC5610731.
Bolli, R. et al. Rationale and design of the CONCERT-HF trial (combination of mesenchymal and c-kit(+) cardiac stem cells as regenerative therapy for heart failure). Circ. Res. 122, 1703-1715, doi:10.1161/CIRCRESAHA.118.312978 (2018).
Brocher, J. Qualitative and quantitative evaluation of two new histogram limiting binarization algorithms. Int. J. Image Process. 8, 30-48 (2014).
Gabisonia, K. et al. MicroRNA therapy stimulates uncontrolled cardiac repair after myocardial infarction in pigs. Nature 569, 418-422, doi:10.1038/s41586-019-1191-6 (2019).
Gyongyosi, M. et al. Porcine model of progressive cardiac hypertrophy and fibrosis with secondary postcapillary pulmonary hypertension. J Transl Med 15, 202, doi:10.1186/s12967-017-1299-0 (2017).
Heallen, T. et al. Hippo signaling impedes adult heart regeneration. Development 140, 4683-4690, doi:10.1242/dev.102798 (2013).
Jones, S. P. et al. The NHLBI-sponsored Consortium for preclinicAl assESsment of cARdioprotective therapies (CAESAR): a new paradigm for rigorous, accurate, and reproducible evaluation of putative infarct-sparing interventions in mice, rabbits, and pigs. Circ Res 116, 572-586, doi:10.1161/CIRCRESAHA.116.305462 (2015).
Lang, R. M. et al. Recommendations for cardiac chamber quantification by echocardiography in adults: an update from the American Society of Echocardiography and the European Association of Cardiovascular Imaging. J Am Soc Echocardiogr 28, 1-39 e14, doi:10.1016/j.echo.2014.10.003 (2015).
Leach, J. P. et al. Hippo pathway deficiency reverses systolic heart failure after infarction. Nature 550, 260-264, doi:10.1038/nature24045 (2017).
Milani-Nejad, N. & Janssen, P. M. Small and large animal models in cardiac contraction research: advantages and disadvantages. Pharmacol Ther 141, 235-249, doi:10.1016/j.pharmthera.2013.10.007 (2014).
McCormick, M. E. et al. Integrated Regional Cardiac Hemodynamic Imaging and RNA Sequencing Reveal Corresponding Heterogeneity of Ventricular Wall Shear Stress and Endocardial Transcriptome. J Am Heart Assoc 5, e003170, doi:10.1161/jaha.115.003170 (2016).
Monroe, T. O. et al. YAP partially reprograms chromatin accessibility to directly induce adult cardiogenesis. Dev. Cell 48, 765-779.e767, doi:10.1016/j.devcel.2019.01.017 (2019).
Morikawa, Y. et al. Actin cytoskeletal remodeling with protrusion formation is essential for heart regeneration in Hippo-deficient mice. Sci Signal 8, ra41, doi:10.1126/scisignal.2005781 (2015).
Riching A S, Song K. Cardiac Regeneration: New Insights Into the Frontier of Ischemic Heart Failure Therapy. Frontiers Bioeng Biotechnology. 2021; 8:637538.
Schindelin, J. et al. Fiji: an open-source platform for biological-image analysis. Nat Methods 9, 676-682, doi:10.1038/nmeth.2019 (2012).
Spannbauer, A. et al. Large Animal Models of Heart Failure with Reduced Ejection Fraction (HFrEF). Front Cardiovasc Med 6, 117, doi:10.3389/fcvm.2019.00117 (2019).
Stubenitsky, R., van der Weerd, R. W., Haitsma, D. B., Verdouw, P. D. & Duncker, D. J. Cardiovascular effects of the novel Ca2+-sensitiser EMD 57033 in pigs at rest and during treadmill exercise. Br J Pharmacol 122, 1257-1270, doi:10.1038/sj.bjp.0701491 (1997).
Tian, Y. et al. A microRNA-Hippo pathway that promotes cardiomyocyte proliferation and cardiac regeneration in mice. Sci Transl Med 7, 279ra238, doi:10.1126/scitranslmed.3010841 (2015).
Vale, P. R. et al. Left ventricular electromechanical mapping to assess efficacy of phVEGF(165) gene transfer for therapeutic angiogenesis in chronic myocardial ischemia. Circulation 102, 965-974, doi:10.1161/01.cir.102.9.965 (2000).
van der Velden, J. et al. Alterations in myofilament function contribute to left ventricular dysfunction in pigs early after myocardial infarction. Circ Res 95, e85-95, doi:10.1161/01.Res.0000149531.02904.09 (2004).
Vela, D., Gahremanpour, A. & Buja, L. M. Method for sectioning and sampling hearts for histologic evaluation after delivery of biological agents by transendocardial injection. Cardiovasc Pathol 24, 304-309, doi:10.1016/j.carpath.2015.04.005 (2015).
Wang, J., Liu, S., Heallen, T. & Martin, J. F. The Hippo pathway in the heart: pivotal roles in development, disease, and regeneration. Nat. Rev. Cardiol 15, 672-684, doi:10.1038/s41569-018-0063-3 (2018).
Xiao, Y. et al. Hippo pathway deletion in adult resting cardiac fibroblasts initiates a cell state transition with spontaneous and self-sustaining fibrosis. Genes Dev 33, 1491-1505, doi:10.1101/gad.329763.119 (2019).

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A method of improving systolic function, the method comprising delivering to heart tissue of a human patient who has experienced myocardial infarction an effective amount of a composition comprising at least one inhibitory nucleic acid, wherein the inhibitory nucleic acid targets Salvador.

2. A method of reducing arrhythmia in a human patient with myocardial fibrosis, the method comprising delivering to heart tissue of the human patient an effective amount of a composition comprising at least one inhibitory nucleic acid, wherein the inhibitory nucleic acid targets Salvador.

3. A method of promoting capillary formation in heart tissue, the method comprising delivering to heart tissue of an individual in need thereof an effective amount of a composition comprising at least one inhibitory nucleic acid, wherein the inhibitory nucleic acid targets Salvador.

4. The method of any preceding claim, wherein the inhibitory nucleic acid has, or is encoded by a sequence having, at least 80% identity to a nucleotide sequence selected from the group consisting of SEQ ID NO: 2 [aagtacgtga agaaggagac g], SEQ ID NO: 3 [aagatttacc ccttcctcct g], and SEQ ID NO: 4 [aattcctgac tggcttcagg t].

5. The method of any preceding claim, wherein the composition comprises (i) an inhibitory nucleic acid having, or encoded by a sequence having, at least 80% identity to SEQ ID NO: 2, (ii) an inhibitory nucleic acid having, or encoded by a sequence having, at least 80% identity to SEQ ID NO: 3, and (iii) an inhibitory nucleic acid having, or encoded by a sequence having, at least 80% identity to SEQ ID NO: 4.

6. The method of any preceding claim, wherein the inhibitory nucleic acid has, or is encoded by a sequence having, at least 85%, 90%, 95%, 96%, 97%, 98%, or 99% identity to a sequence selected from the group consisting of SEQ ID NO: 2, SEQ ID NO: 3, and SEQ ID NO: 4.

7. The method of any preceding claim, wherein the inhibitory nucleic acid has a sequence, or is encoded by a sequence, selected from the group consisting of SEQ ID NO: 2, SEQ ID NO: 3, and SEQ ID NO: 4.

8. The method of any one of claims 1 to 7, wherein the inhibitory nucleic acid is an antisense DNA molecule.

9. The method of any one of claims 1 to 7, wherein the inhibitory nucleic acid is an RNA.

10. The method of claim 9, wherein the inhibitory nucleic acid is a short hairpin RNA (shRNA).

11. The method of claim 10, wherein the shRNA is at least 43 nucleotides in length.

12. The method of claim 10, wherein the shRNA is less than 138 nucleotides in length.

13. The method of claim 10, wherein the shRNA comprises a loop structure of between 5 and 19 nucleotides in length.

14. The method of any one of claims 10 to 13, wherein a nucleotide sequence encoding the RNA is comprised in a nucleic acid construct, and wherein the RNA is expressed in cardiomyocytes.

15. The method of claim 14, wherein the nucleotide sequence encoding the RNA is operably linked to a tissue-specific promoter.

16. The method of claim 15, wherein the promoter is a cardiac troponin T promoter.

17. The method of claim 15, wherein the nucleic acid construct comprises a post-transcriptional regulatory element.

18. The method of claim 17, wherein the post-transcriptional regulatory element is a woodchuck hepatitis virus post-transcriptional regulatory element (WPRE).

19. The method of any preceding claim, wherein a nucleotide sequence encoding the inhibitory nucleic acid is comprised in a vector.

20. The method of claim 19, wherein the vector is a non-viral vector.

21. The method of claim 19, wherein the vector is a non-integrating vector.

22. The method of claim 19, wherein the vector is a viral vector.

23. The method of claim 22, wherein the vector is an adeno-associated viral (AAV) vector.

24. The method of claim 22, wherein the vector is a lentiviral vector.

25. The method of claim 5, wherein nucleotide sequences encoding the inhibitory nucleic acids are comprised in a single nucleic acid construct, and wherein the nucleotide sequences are expressed in cardiomyocytes.

26. The method of claim 25, wherein the nucleotide sequences encoding the inhibitory nucleic acids are regulated by a single promoter.

27. The method of any one of claims 1 to 26, wherein the composition comprises a nucleic acid construct comprising: (i) a nucleic acid having the nucleotide sequence set forth in SEQ ID NO: 2, (ii) a nucleic acid having the nucleotide sequence set forth in SEQ ID NO: 3, and (iii) a nucleic acid having the nucleotide sequence set forth in SEQ ID NO: 4; and wherein nucleic acids (i)-(iii) are operably linked to a promoter.

28. The method of claim 27, wherein the promoter is a cardiac troponin T promoter.

29. The method of claim 27 or 28, wherein the nucleic acid construct comprises sequences encoding a 3′ microRNA-30 sequence and a 5′ microRNA-30 sequence.

30. The method of any one of claims 27 to 29, wherein the nucleic acid construct is comprised in a viral vector.

31. The method of claim 30, wherein the vector is an adeno-associated viral (AAV) vector.

32. The method of claim 30, wherein the vector is a lentiviral vector.

33. The method of claim 30, wherein the nucleic acid construct comprises a post-transcriptional regulatory element.

34. The method of claim 33, wherein the post-transcriptional regulatory element is a woodchuck hepatitis virus post-transcriptional regulatory element (WPRE).

35. The method of any one of claims 30 to 34, wherein the nucleic acid construct comprises 5′ and 3′ inverted terminal repeats.

36. The method of claim 2, wherein the human patient has experienced myocardial infarction.

37. The method of claim 2, wherein the arrhythmia is atrial arrhythmia.

38. The method of claim 2, wherein the arrhythmia is ventricular arrhythmia.

39. The method of claim 3, wherein the individual has experienced myocardial infarction.

40. An inhibitory nucleic acid for use in a method of improving systolic function in a human patient who has experienced myocardial infarction, the method comprising delivering to heart tissue of the human patient an effective amount of a composition comprising at least one inhibitory nucleic acid that targets Salvador.

41. An inhibitory nucleic acid for use in a method of reducing arrhythmia in a human patient with myocardial fibrosis, the method comprising delivering to heart tissue of the human patient an effective amount of a composition comprising at least one inhibitory nucleic acid that targets Salvador.

42. An inhibitory nucleic acid for use in a method of promoting capillary formation in heart tissue, the method comprising delivering to heart tissue of an individual in need thereof an effective amount of a composition comprising at least one inhibitory nucleic acid that targets Salvador.

43. A method of treating, preventing, or reducing the risk of arrhythmia in a human patient, the method comprising
(a) identifying a need for treatment, prevention, or reduction of the risk of arrhythmia in a human patient; and
(b) delivering to heart tissue of the human patient an effective amount of a composition comprising at least one inhibitory nucleic acid, wherein the inhibitory nucleic acid targets Salvador.

44. The method of claim 43, wherein the human patient has experienced myocardial infarction or is at risk for myocardial infarction.
